(19) United States
(12) Patent Application Publication
(10) Pub. No.: US 2022/0031615 A1
(43) Pub. Date: Feb. 3, 2022

(54) VESICLES

(71) Applicant: SEQUESSOME TECHNOLOGY HOLDINGS LIMITED, Valletta (MT)

(72) Inventors: Richard Wolf Garraway, Greater London (GB); William Henry, Greater London (GB)

(21) Appl. No.: 17/181,796

(22) Filed: Feb. 22, 2021

Related U.S. Application Data
(63) Continuation of application No. 14/908,494, filed on Jan. 28, 2016, now abandoned.

(30) Foreign Application Priority Data
Jul. 31, 2013 (GB) 1313734.4
Jul. 31, 2013 (GB) 1313735.1

(51) Int. Cl. A61K 9/127; A61K 8/55; A61K 9/00; A61K 47/24; A61K 8/14; A61K 8/67; A61K 9/107; A61K 8/39; A61K 8/64; A61Q 19/00; A61K 47/54; A61K 31/664; A61K 47/32

(52) U.S. Cl. CPC A61K 9/127; A61K 2800/10; A61K 9/0014; A61K 47/24; A61K 8/14; A61K 9/1271; A61K 8/676; A61K 9/107; A61K 8/39; A61K 8/64; A61Q 19/00; A61K 47/549; A61K 47/542; A61K 31/664; A61K 47/32; A61K 8/553

(57) ABSTRACT

The present invention relates to vesicular formulations for use in the topical administration of a therapeutic, metabolic, cosmetic or structural Agent Of Interest (“AOI”) and methods of administering an AOI.

This Application is a continuation of U.S. application Ser. No. 14/908,494 filed Jan. 28, 2016, which is the National Stage of International Application No. PCT/EP2014/066545, filed Jul. 31, 2014, which claims the benefit of and priority to GB Application No. 1313735.1, filed Jul. 31, 2013, and GB Application No. 1313734.4, filed Jul. 31, 2013. The entire contents of all of which are hereby incorporated by reference.

The present invention relates to vesicular formulations for use in the topical administration of a therapeutic, metabolic, cosmetic or structural Agent Of Interest (“AOI”) and methods of administering an AOI.

U.S. Pat. No. 6,165,500 describes a preparation for the application of agents which are provided with membrane-like structures consisting of one or several layers of amphiphilic molecules, or an amphiphilic carrier substance, in particular for transporting the agent into and through natural barriers such as skin and similar materials. These Transfersomes™ consist of one or several components, most commonly a mixture of basic substances, one or several edge-active substances, and agents.

US Patent Application Publication No. US 2004/0071767 describes formulations of nonsteroidal anti-inflammatory drugs (NSAIDs) based on complex aggregates with at least three amphiphatic components suspended in a pharmaceutically acceptable medium.

US Patent Application Publication No. US 2004/0105881 describes extended surface aggregates, suspendable in a suitable liquid medium and comprising at least three amphiphats (amphiphatic components) and being capable to improve the transport of actives through semi-permeable barriers, such as the skin, especially for the non-invasive drug application in vivo by means of barrier penetration by such aggregates. WO 2010/140061 describes the use of “empty” vesicular formulations for the treatment of deep tissue pain. WO 2011/022707 describes the use of other formulations of “empty” vesicles for treating disorders relating to fatty acid deficiencies and inter alia disorders related to inflammation. Vesicular formulations to which therapeutic entities can be attached are described in WO2011/022707 and WO2010/140061.

These documents neither disclose or teach vesicular formulations for the use in the topical administration of an AOI, nor that an AOI may be covalently bonded to a component of the vesicle such that the majority of the AOI is external to the vesicle. Citation of any reference in this section of the application is not an admission that the reference is prior art to the invention. The above noted publications are hereby incorporated by reference in their entirety.

Liposomal vesicles have been used in the past in attempts to deliver active compounds (AOIs) into the body.

Flexible forms of liposomes (“Transfersomes®”) are vesicles made from a combination of a fat (for example, soy phosphatidylcholine) and a fatty acid or surfactant (for example, Tween) that can pass through the skin surface. The polyethylene glycol (“PEG”) in the surfactant of these vesicles is hygroscopic and penetrates skin pores along a water gradient. These vesicles have been tested as vehicles for transporting other AOIs into the body via the transdermal route, either by placing the AOI to be transported inside the lumen of the vesicle or incorporating the AOI into the membrane of the vesicle, as one of the membrane components.

Either of these methods must rely on some form of disruption of the vesicle in order to release the AOI.

Further, there are products that one might wish to transport through the skin which are either too large to be incorporated into the Transfersome in this way or which possess a chemistry that is incompatible with the normal chemistry of these vesicles.

Further, where some of the PEG-containing surfactant components are replaced with the AOI, this affects the flexibility of the vesicle and removes some of the motive power.

The current invention circumvents these problems by physically attaching an AOI to the vesicle, so that the vesicle acts purely as a mechanical device, pulling the desired AOI beneath the skin's surface.

These vesicles of the invention can be used for transporting other moieties/AOI into the body via the transdermal route, by attaching such moieties or AOI to a component of the vesicle, such that the AOI lies outside the vesicle.

Accordingly, the present invention provides, in a first aspect, a vesicular formulation comprising a lipid, a surfactant and an AOI, wherein the AOI is bonded to a component of the vesicle such that at least a portion of the AOI is on the external surface of the vesicle, and is external to the vesicle membrane. Preferably, the component to which the AOI is bonded is a lipid and/or a surfactant component. At least a portion means that of the total AOI that is external to the vesicle at least 5%, 10% or 20%, suitably 40%, or more than 50% of each molecule (in terms of size or volume of the molecule) is external to the membrane of the transfersome. Preferably the majority of the AOI, more preferably the entire AOI molecule is external to the vesicle. The AOI may be covalently bonded to a component such that it presents on the external surface of the vesicle.

By the AOI that is external to the vesicle, it is meant those AOI that are ‘facing outwards’. During manufacture of the vesicles, whereby the surfactant or lipid component that is bonded to the AOI is mixed with the unmodified components, the orientation of the modified molecule cannot be controlled. Thus, approximately 50% of the molecules to which the AOI is attached will be in the ‘incorrect’ orientation, meaning that a portion of the AOI will be present in the lumen of the vesicle. Of the modified molecules that are in the “correct” orientation such that the AOI is external to the vesicle, at least 50% of the AOI molecule itself, in terms of physical size/volume, is external to the vesicular membrane. The manufacturing process may result in a lower proportion of the AOI being external to the vesicle i.e. the external concentration of the AOI may be between 1% to 10% (including 2%, 3%, 4%, 5%, 6%, 7%, 8% or 9%) 10% to 50%, 15% to 45%, 20% to 40% or 25% to 30% (wt/vol) of the total AOI in the formulation. By external concentration it is meant the concentration of AOI that is available for release and/or to exert its therapeutic activity once the vesicles have penetrated the skin.

The benefits of the vesicular formulation of the invention relates to the speed, depth and amount of AOI and the size and nature of that AOI that penetrates the skin, when the AOI is bonded to the vesicle and topically applied.

The formulation may be a cream, lotion, ointment, gel, solution, spray, lacquer, mousse or film forming solution.

The vesicular formulation may or may not contain any known therapeutic agent, other than the AOI bonded to the vesicles. The vesicular formulation comprising an AOI may or may not be free of any further biologically active or pharmaceutically active product. A biologically active or pharmaceutically active agent is here defined as an agent that has pharmacological, metabolic or immunological activity.

The invention encompasses vesicular formulations comprising one or more phospho or sulpholipids and one or more surfactants that are effective for the delivery of an AOI. The surfactant may be non-ionic.

The vesicular formulation of the invention is able (without wishing to be bound by theory) to achieve its function through the unique properties of vesicles, which are bilayer vesicles composed of surfactant and lipid, such as soy phosphatidylcholine. The uniqueness of the vesicles derives from the inclusion in the formulation of a specific amount of non-ionic surfactant, which modifies the phospholipid membrane to such an extent that the resulting vesicles are in a permanent liquid crystalline state and, since the surfactant also confers membrane stability, the vesicles are ultra deformable and stable (have reduced rigidity without breaking).

The vesicular formulation comprises/forms into vesicles suspended in, for example, an aqueous buffer that is applied topically. The vesicles of the vesicular formulation comprise a bilayer or unilamellar membrane, surrounding an empty core. They range in size from 60 nm in diameter to 200 nm in diameter, and may range from 100 nm to 150 nm in diameter. The vesicles are highly hydrophilic and this property, together with their ultra deformability, is key to their ability to be transported across the skin. When the formulation of the invention is applied to the skin and allowed to dry, the rehydration driving force of the vesicles combined with their deformability gives rise to movement of the vesicles to areas of higher water content on and below the skin permeability barrier. This drives their movement through skin pores and intracellular gaps. The specific ratio of surfactant to non-ionic surfactant facilitates transdermal delivery of vesicles. The movement of the vesicles through the pores and intracellular gaps carry or pull with them the AOI.

Once they pass through the skin, the vesicles of the invention eventually present as intact vesicles. Efficient clearance of vesicles does not occur via the cutaneous blood microvasculature (capillaries) owing to their relatively large size, but they are hypothesised to be transported with the interstitial fluid into other and/or deeper tissues below the site of dermal application. A preclinical study conducted with vesicles of the invention labelled with a marker molecule (ketoprofen) showed that the vesicles did not enter the vasculature because, following topical application, high concentrations of the marker molecule were observed locally with low systemic absorption.

The AOI may be bonded to a surfactant component or to a lipid component of a vesicle. Alternatively, both a lipid component and a surfactant component of a vesicle may have an AOI bonded to them.

A vesicle of the formulation may have a single or a plurality of AOIs bonded to its external surface. Wherein a plurality of AOIs are bonded, the AOIs may all be the same, i.e. homogenous, or the AOIs may be different, i.e. heterogeneous.

The AOI may be an element, an ion, a small molecule, a carbohydrate, a lipid, an amino acid, a peptide, a protein, a macromolecule or a macrocyclic molecule. The AOI may be a micronutrient.

The AOI may be a skin structural protein (such as elastin or collagen), a therapeutic protein, porphyrin or chromophore containing macromolecule, a vitamin, titanium dioxide, zinc oxide, melanin or a melanin analogue. The AOI may be a peptide or an anti-inflammatory drug, such as an NSAID. Specifically, the AOI may be tetrapeptide-7, tripeptide 1, ascorbic acid, Naproxen or Diclofenac.

The AOI to be bonded may be covalently or otherwise bonded directly to either the phospholipid or surfactant component of the vesicle or the lipid component of the vesicle. It may be desirable to use a link or bridge that is covalently or otherwise bonded to both the fatty acid, surfactant or lipid component and the AOI. In one example, if an inorganic AOI were to be added (for example a metal salt or oxide), an additional linker, for example a metal chelating agent such as EDTA might first be conjugated to the vesicle component. In another example it may be desirous to use a longer molecule, for example a polymer (such as polyethylene glycol; PEG), to facilitate the efficacy of the bonding process and/or the effectiveness of the bound AOI. Such linkers/longer bridging molecules will be particularly of benefit when it is desirable to hold an AOI at such a distance from the vesicles in order to prevent it interfering with the membrane itself. This may occur if the AOI is particularly hydrophobic.

Large molecules or macromolecules may be covalently bonded to the vesicle component(s). Examples include structural skin proteins such as collagen and elastin; therapeutic proteins; and enzymes.

A plurality of AOIs may be bonded to a lipid or surfactant component to present on the external surface of the vesicle so that once taken through the skin, they continue to present on the surface of the vesicle. Examples include anti-oxidants; vitamins; inorganic compounds such as TiO₂ and ZnO; porphyrin molecules for use in photodynamic therapies.

Transporting vitamins into the body via skin, may either replace missing vitamin generating capability (for example, vitamin D), enhance the skin's (or any other organ's) ability to protect and repair itself (for example, vitamins C and E), or treat dermal or other conditions such as seborrhoeic dermatitis (for example vitamin B₇). The reference to skin includes the general skin of the body and any other external integument, such as the epithelium of the ear, nose, throat and eye, including the sclera of the eye, and other mucosal membranes, such as the vagina and anus/rectum.

Vitamin D is actually a group of fat-soluble compounds responsible for enhancing intestinal absorption of calcium and phosphate. The most important of this group are D₃ (choleclaciferol) and D₂ (ergocalciferol). Vitamin D deficiency causes osteomalacia (rickets in children) and low levels have been associated with low bone mineral density.

Mammalian skin makes vitamin D₃ through the action of UV radiation on its precursor, 7-dehydrocholesterol, and supplies about 90 percent of our vitamin D. Sunscreen absorbs ultraviolet light and prevents it from reaching the skin. It has been reported that sunscreen with a sun protection factor (SPF) of 8 based on the UVB spectrum can decrease vitamin D synthetic capacity by 95 percent, whereas sunscreen with an SPF of 15 can reduce synthetic capacity by 98 percent.

More recently there has been a trend toward increased use of higher SPF sunscreens (between 25 SPF and 50 SPF) and complete sunblock products as public awareness of the dangers of tanning has grown. In addition, both cosmetic skincare and colour cosmetic products have had sunscreens of 15, 20 and 25 SPF added to their formulation to provide a degree of sun protection.

When the formulation comprises vitamin D₃ (which is not to say that it also does not comprise vitamin C and/or E and/or B₇) the formulation may be incorporated into a sunscreen product, a sun block product, an after-sun product or other skincare or cosmetic product to supplement low vitamin D levels. Low vitamin D levels may be caused by low light conditions, or use of sunblock, which can prevent the manufacture of vitamin D within the body.

Thus, the present invention may associate vitamin D₃/choleclaciferol with a flexible transdermal vesicle i.e. a formulation comprising a lipid and surfactant, by tethering the vitamin to its external surface. The resulting formulation can then be included in sunscreens, after-sun formulations and cosmetic products that include sunscreen. This “vesicle/vitamin D combination” will penetrate the skin and deliver its payload to the stratum basale and stratum spinosum layers in the epidermis.

In the body, cholecalciferol (vitamin D₃) is first converted to calcidiol in the liver. Circulating calcidiol is then coverted into calcitriol, the biologically active form of vitamin D, in the kidneys. Low blood calcidiol (25-hydroxy-vitamin D) can result from avoiding the sun. The invention therefore includes associating either calcidiol or calcitriol with a transdermal vesicle, by way of bonding or tethering to a vesicle component.

Certain other vitamins for example vitamin C and vitamin E, have important anti-oxidant properties and this has seen them be incorporated into skincare products to reduce the signs of aging and skin damage.

The most biologically active form of vitamin E is the fat soluble α-tocopherol and one embodiment of the current invention anticipates associating α-tocopherol with a vesicle component for incorporation into skincare preparations, including sunscreens and after-sun products to ameliorate sun damage.

Vitamin C (water-soluble ascorbate) is a cofactor in many enzymatic reactions including several collagen synthesis reactions. These reactions are important in wound healing and in preventing bleeding from capillaries. Therefore, the current invention includes associating ascorbate with a vesicle component, for incorporation both into skincare and suncare preparations to ameliorate damage to collagen, and for incorporation into wound care products.

Thus, when the micronutrient comprises vitamin C and/or vitamin E, the formulation may be incorporated into a sunscreen product, a sun block product, an after-sun product or other skincare or cosmetic product to supplement epidermal and dermal vitamin C and/or vitamin E and prevent or assist in the repair of sun-damaged or aging skin.

Vitamin B₇ (water-soluble Biotin) is a co-enzyme for carboxylase enzymes. A deficiency in biotin can cause a dermatitis in the form of a rash. In addition patients with phenylketonuria (an inability to break down phenylalanine) exhibit forms of eczema and seborrhoeic dermatitis that can be ameliorated by increasing dietary biotin.

Thus, when the micronutrient comprises vitamin B₇, the formulation may be incorporated into a sunscreen product, a sun block product, an after-sun product or other skincare or cosmetic product to supplement epidermal and dermal vitamin B₇ and ameliorate the dermatoses associated with a deficiency of this vitamin.

Peptides, such as tetrapeptide-7 or tripeptide-1, may be bonded to a fatty acid or surfactant component of the vesicle membrane. Tetrapeptide-7 may be useful in fighting inflammation and act to stimulate skin regeneration by way of collagen production. This means that it is particularly useful in skin care, and anti-ageing products. Tripeptide-1 has a similar action. Efficient delivery through the external layer of skin may provide increased effects at lower levels/concentrations thus minimising possible side effects resulting from the suppression of interleukins.

Non-steroidal anti-inflammatory drugs (NSAIDs) are painkilling agents generally used to relieve the symptoms of osteoarthritis, sports associated joint pain, back pain, headaches and dental pain. Examples of NSAIDs are aspirin, ibuprofen, diclofenac and naproxen. Again, effective and efficient delivery of such drugs directly to the site of pain and inflammation may result in the use of lower and/or targeted dosages and thus the reduction or elimination of side effects, such as gastrointestinal problems, renal problems and cardiac problems.

The invention may include bonding a larger number of small, inactive AOIs to the surface of the vesicle, so that once under the skin it becomes anchored and the longevity of the benefits of the presence of the vesicle itself, for example water retaining, structure supporting, can be extended.

The AOI may be bonded (or attached or tethered) to the surfactant component of the vesicle. The bonding to the surfactant may be directly onto the surfactant by ester bond if the molecule has a hydroxyl group. An alternative method of bonding is to substitute an atom or functional group of the surfactant (for example in the case of Tween, a polyethylene glycol polymer) with the AOI. A third method of bonding is directly to a fatty acid, optionally via an ester bond. If the AOI is an inorganic molecule then a further linking molecule can first be conjugated to the vesicle component, for example a metal chelating agent such as EDTA in the case of a metal salt. If it is desirous that the AOI be held at some distance from the vesicle in order to maximise its efficiency (for example to expose an active site on an AOI that is an enzyme), then a linking molecule, for example a polymer chain (for example polyethylene glycol) may be bonded to both a component of the vesicle and the AOI.

The AOI may be attached (bonded or tethered) to the lipid component of the vesicle. The bonding to the lipid might be achieved via any of the glycerol hydroxyl groups by an ester bond, for example by eliminating a fatty acid and replacing with the AOI. Alternatively, the method of attachment may be by replacement of the phosphatidyl moiety such that the final molecule has two fatty acid chains together with the tethered AOI. The modified lipid inserts in the aliphatic region as normal and with the free rotation available on the glycerol template, the tethered AOI would locate on the outside of the vesicle. An amide bond may be used for a more stable alternative, should the AOI be required to be tethered to the vesicle for a longer duration. This may be desirable, for example, if the target for the AOI is deep tissue, such as joints, rather than the upper dermal layers. A combination of less stable and more stable bonds may be used (e.g. ester and amide, respectively) to achieve staggered release of the AOI.

The method of bonding to any component may be hydrolysable or non-hydrolysable. If it is desirable that the AOI should be detached once within or under the skin, the link should be hydrolysable. If it is desirable that the bonded AOI should remain bound to the vesicle once within or under the skin, the link should be non-hydrolysable.

The AOI may be covalently bonded or conjugated to a membrane component; the bond may be hydrophilic or hydrophobic or hydrostatic; The bond may be a hydrogen bond, an ionic bond.

The terms “bonding”, “attaching” and “tethering” are used herein throughout interchangeably to encompass all the bonds mentioned above.

The present invention can be used to administer an AOI to the skin of a mammal. Any mammal can be included, including humans, dogs, cats, horses, food production animals and pets. The AOI may be a therapeutic entity or a cosmetic entity or a non-therapeutic or non-cosmetic entity, alternatively or in addition the AOI may be metabolic and/or structural.

Accordingly, a second aspect of the invention provides a vesicular formulation comprising a lipid, a surfactant and an AOI, wherein the AOI is bonded or attached to a component of the vesicle such that the majority of the AOI that is external to the vesicle. for use in delivering the AOI through the skin of a subject, wherein the formulation is topically applied.

A third aspect of the invention provides a method of delivering an AOI through the skin of a subject, the method comprising topically applying to the skin of the patient the vesicular formulation of the invention in an amount sufficient to penetrate the skin to deliver the AOI.

The invention also provides a method of delivering more than one AOI through the skin of the patient, the method comprising topically applying either the vesicular formulation of the invention where the vesicles have a heterogeneous plurality of AOIs bonded to them and/or applying the vesicular formulation of the invention where the formulation is a blend of vesicles, each formulation having vesicles which have different single or homogenous plurality of AOIs bonded to them.

The lipid in the vesicular formulations may be a phospholipid. A second lipid may be present, which may be a lysophospholipid. The lipid may be a sulpholipid. The surfactant may be a non-ionic surfactant.

The formulations of the invention form vesicles or other extended surface aggregates (ESAs), wherein the vesicular preparations have improved permeation capability through the semi-permeable barriers, such as skin. The size of the vesicle prevents penetration into the vasculature and as a result prevents systemic delivery. While not to be limited to any mechanism of action, the formulations of the invention are able to form vesicles characterized by their deformability and/or adaptability.

The specific composition of the vesicular formulation will determine to which layer of the skin the AOI can be delivered. Certain formulations will penetrate only the upper layers of the skin whilst other formulations will travel to deeper layers. The vesicular formulation will be chosen depending on the AOI to be delivered. For example, if collagen is the AOI to be delivered deep into the skin, it will be attached to vesicular formulation that is able to penetrate the deeper layers of the skin.

As a fourth aspect, the invention provides a method of making a vesicular formulation in accordance with the first to third aspects of the invention. The method comprises attaching an AOI to a vesicular component, mixing the AOI/component with an unmodified phospholipid and surfactant to form the vesicular formulations of the invention.

A fifth aspect of the invention relates to the vesicular formulation of the first aspect for use in the treatment of disease. The disease to be treated will depend upon the AOI that is tethered to the vesicles.

The invention provides a vesicular formulation in accordance with the first aspect, wherein the AOI is a vitamin, such as vitamin C, vitamin E, vitamin D or vitamin A, for use in a skin care product, for use in an anti-ageing product or for use in a sun protection (UV protection) product.

Also provided is a vesicular formulation in accordance with the invention, wherein the AOI is a peptide, such as tetra-peptide 7 or tri-peptide 1, for use in anti-ageing products, for use in encouraging or boosting collagen production, or for use in cosmetics.

Also provided is a vesicular formulation in accordance with the invention, wherein AOI is an NSAID, such as Naproxen or Diclofenac, for use in the treatment of osteoarthritis, for use in the treatment of arthritic joint pain, for use in the treatment of muscle pain, for use in the treatment of muscle strain or for use in the treatment of inflammation.

As will be appreciated, other AOIs may be tethered to the vesicles of the invention in order to treat a wide variety of diseases.

All features of the first aspect of the invention apply to the second to fifth aspects mutatis mutandis.

During the manufacture of the vesicles, the ratio of modified components (i.e. with AOIs attached) to non-modified components (i.e. without AOIs attached) is adjusted to control both the degree with which multiple modified components (and thus AOIs) are incorporated into the vesicles and also the number of vesicles that contain at least one AOI. Where a proportion of unmodified vesicles remain in the final preparation, these will complement the “pulling” action of the modified forms by following these into the skin pores and “pushing” from behind. The percentage of modified vesicles (as a proportion of total vesicles) in the final preparation may range from 0.1% to 100%, or from 1% to 100%, from 10% to 90%, from 25% to 75% or 50%.

To ensure that a high proportion of vesicles has the desired AOI attached, or has multiple AOIs attached, 100% modified surfactant may be used to mix with the lipid (or vice versa). At the other end of the scale, for a more dilute effect, where only a few vesicles have an AOI attached or only a single AOI is attached to the vesicles, for example, a blend of 5% modified to 95% unmodified surfactant is used. Generally, however between 80% and 10% of the lipid or surfactant is replaced with a modified lipid or surfactant component. Between 75% and 15% of the lipid or surfactant component may be replaced. Suitably, between about 70%, 65%, 60%, 50%, 40%, 35%, 30%, 25%, 20%, 15% or 10% or any range between these values, of either the lipid component or the surfactant component may be replaced with a modified lipid or modified surfactant component, bonded to the AOI, respectively A proportion of both the lipid and the surfactant components may be replaced. Further refinement can be carried out by extracting the modified vesicles and mixing them into a precise “dose” with pure unmodified vesicles, or by mixing with vesicles modified with a different AOI.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

As used herein, a “sufficient amount,” “amount effective to” or an “amount sufficient to” achieve a particular result refers to an amount of the formulation of the invention that is effective to produce a desired effect, which is optionally a therapeutic effect (i.e., by administration of a therapeutically effective amount). Alternatively stated, a “therapeutically effective” amount is an amount that provides some alleviation, mitigation, and/or decrease in at least one clinical symptom. Clinical symptoms associated with the disorder that can be treated by the methods of the invention are well-known to those skilled in the art. Further, those skilled in the art will appreciate that the therapeutic effects need not be complete or curative, as long as some benefit is provided to the subject.

As used herein, the terms “treat”, “treating” or “treatment” of mean that the severity of a subject's condition is reduced or at least partially improved or ameliorated and/or that some alleviation, mitigation or decrease in at least one clinical symptom is achieved and/or there is an inhibition or delay in the progression of the condition and/or delay in the progression of the onset of disease or illness. The terms “treat”, “treating” or “treatment of” also means managing the disease state. “Prevention” means prophylactic treatment.

As used herein, the term “pharmaceutically acceptable” when used in reference to the formulations of the invention denotes that a formulation does not result in an unacceptable level of irritation in the subject to whom the formulation is administered. Preferably such level will be sufficiently low to provide a formulation suitable for approval by regulatory authorities.

As used herein with respect to numerical values, the term “about” means a range surrounding a particular numeral value which includes that which would be expected to result from normal experimental error in making a measurement. For example, in certain embodiments, the term “about” when used in connection with a particular numerical value means +−20%, unless specifically stated to be +−1%, +−2%, +−3%, +−4%, +−5%, +−10%. +−15%, or +−20% of the numerical value.

The formulation of the invention provided herein comprises at least one lipid, preferably a phospho or sulpholipid, at least one surfactant, preferably a nonionic surfactant, optionally suspended in a pharmaceutically acceptable medium, preferably an aqueous solution, preferably having a pH ranging from 3.5 to 9.0, preferably from 4 to 7.5. The formulation of the invention may optionally contain buffers, antioxidants, preservatives, microbicides. antimicrobials, emollients, co-solvents, and/or thickeners. The formulation of the invention may comprise a mixture of more than one lipid, preferably more than one phospholipid. The formulation of the invention may consist essentially of at least one lipid, preferably a phospholipid, at least one surfactant, preferably a nonionic surfactant, a pharmaceutically acceptable carrier, and optionally buffers, antioxidants, preservatives, microbicides, antimicrobials, emollients, co-solvents, and/or thickeners. The formulation of the invention may consist of at least one lipid, preferably a phospholipid, at least one surfactant, preferably a nonionic surfactant, a pharmaceutically acceptable carrier, and one or more of the following: buffers, antioxidants, preservatives, microbicides, antimicrobials, emollients, co-solvents, and thickeners.

Table 1 lists preferred phospholipids in accordance with the invention.

TABLE 1

Bechen(o)yl
Eruca(o)yl
Arachin(o)yl
Gadolen(o)yl
Arachindon(o)yl
Ole(o)yl
Stear(o)yl
Linol(o)yl
Linole(n/o)yl
Palmitole(o)yl
Palmit(o)yl
Myrist(o)yl
Laur(o)yl
Capr(o)yl

The preferred lipids in the context of this disclosure are uncharged and form stable, well hydrated bilayers; phosphatidylcholines, phosphatidylethanolamine, and sphingomyelins are the most prominent representatives of such lipids. Any of those can have chains as listed in the Table 1; the ones forming fluid phase bilayers, in which lipid chains are in disordered state, being preferred.

Different negatively charged, i.e., anionic, lipids can also be incorporated into vesicular lipid bilayers. Attractive examples of such charged lipids are phosphatidylglycerols, phosphatidylinositols and, somewhat less preferred, phosphatidic acid (and its alkyl ester) or phosphatidylserine. It will be realized by anyone skilled in the art that it is less commendable to make vesicles just from the charged lipids than to use them in a combination with electro-neutral bilayer component(s). In case of using charged lipids, buffer composition and/or pH care must selected so as to ensure the desired degree of lipid head-group ionization and/or the desired degree of electrostatic interaction between the, oppositely, charged drug and lipid molecules. Moreover, as with neutral lipids, the charged bilayer lipid components can in principle have any of the chains of the phospholipids as listed in the Table 1. The chains forming fluid phase lipid bilayers are clearly preferred, however, both due to vesicle adaptability increasing role of increasing fatty chain fluidity and due to better ability of lipids in fluid phase to mix with each other.

The fatty acid- or fatty alcohol-derived chain of a lipid is typically selected amongst the basic aliphatic chain types below:

| | | |
|---|---|---|
| Dodecanoic | cis-9-Tetradecanoic | 10-cis,13-cis-Hexadecadienoic |
| Tridecanoic | cis-7-Hexadecanoic | 7-cis,10-cis-Hexadecandienoic |
| Tetradecanoic | cis-9-Hexadecanoic | 7-cis,10-cis,13-cis- |
| | | Hexadecatrienoic |
| Pentadecanoic | cis-9-Octadecanoic | 12-cis,15-cis-Octadecadienoic |
| Hexadecanoic | cis-11-Octadecanoic | trans-10,trans-12-Octadecadienoic |
| Heptadecanoic | cis-11-Eicosanoic | 9-cis,12-cis,15-cis- |
| | | Octadecatrienoic |
| Octadecanoic | cis-14-Eicosanoic | 6-cis,9-cis,12-cis-Octadecatrienoic |
| Nonadecanoic | cis-13-Docosanoic | 9-cis,11-trans,13-trans- |
| | | Octadecatrienoic |
| Eicosanoic | cis-15-Tetracosanoic | 8-trans,10-trans,12-cis- |
| | | Octadecatrienoic |
| Heneicosanoic | trans-3- | 6,9,12,15-Octadecatetraenoic |
| | Hexadecanoic | |
| Docosanoic | tans-9-Octadecanoic | 3,6,9,12-Octadecatetraenoic |
| Tricosanoic | trans-11- | 3,6,9,12,15-Octadecapentaenoic |
| | Octadecanoic | |
| Tetracosanoic | | 14-cis,17-cis-Eicosadienoic |
| | | 11-cis,14-cis-Eicosadienoic |
| | | 8-cis,11-cis-14-cis-Eicosadienoic |
| | | 8-cis,11-cis-14-cis-Eicosadienoic |
| | | 5,8,11all-cis-Eicosatrienoic |
| | | 5,8,11; 14-all-cis-Eicosatrienoic |
| | | 8,11,14,17-all-cis-Eicosatetraenoic |
| | | 5,8,11,14,17-all-cis- |
| | | Eicosatetraenoic |
| | | 13,16-Docosadienoic |
| | | 13,16,19-Docosadienoic |
| | | 10,13,16-Docosadienoic |
| | | 7,10,13,16-Docosadienoic |
| | | 4,7,10,13,16-Docosadienoic |
| | | 4,7,10,13,16,19-Docosadienoic |

Other double bond combinations or positions are possible as well.

A preferred lipid of the invention is, for example, a natural phosphatidylcholine, which used to be called lecithin. It can be obtained from egg (rich in palmitic, C16:0, and oleic, C18:1, but also comprising stearic, C18:0, palmitoleic, C16:1, linolenic, C18:2, and arachidonic, C20:4(M, radicals), soybean (rich in unsaturated C18 chains, but also containing some palmitic radical, amongst a few others), coconut (rich in saturated chains), olives (rich in monounsaturated chains), saffron (safflower) and sunflowers (rich in n-6 linoleic acid), linseed (rich in n-3 linolenic acid), from whale fat (rich in monounsaturated n-3 chains), from primrose or primula (rich in n-3 chains). Preferred, natural phosphatidyl ethanolamines (used to be called cephalins) frequently originate from egg or soybeans. Preferred sphingomyelins of biological origin are typically prepared from eggs or brain tissue. Preferred phosphatidylserines also typically originate from brain material whereas phosphatidylglycerol is preferentially extracted from bacteria, such as E. coli, or else prepared by way of transphosphatidylation, using phospholipase D, starting with a natural phosphatidylcholine. The preferably used phosphatidylinositols are isolated from commercial soybean phospholipids or bovine liver extracts. The preferred phosphatidic acid is either extracted from any of the mentioned sources or prepared using phospholipase D from a suitable phosphatidylcholine.

Furthermore, synthetic phosphatidylcholines may be used.

The amount of lipid in the formulation is from about 1% to about 12%, about 1% to about 10%, about 1% to about 4%, about 4% to about 7% or about 7% to about 10% by weight. The lipid may be a phospholipid. The phospholipid may be a phosphatidylcholine.

The lipid in the formulation may not comprise an alkyl-lysophospholipid. The lipid in the formulation may not comprise a polyeneylphosphatidylcholine.

The term “surfactant” has its usual meaning. A list of relevant surfactants and surfactant related definitions is provided in EP 0 475 160 A1 (see, e.g., p. 6, 1. 5 to p. 14. 1.17) and U.S. Pat. No. 6,165,500 (see, e g., col. 7, 1. 60 to col. 19, 1. 64), each herein incorporated by reference in their entirety, and in appropriate surfactant or pharmaceutical Handbooks, such as Handbook of Industrial Surfactants or US Pharmacopoeia, Pharm. Eu. In some embodiments, the surfactants are those described in Tables 1-18 of U.S. Patent Application Publication No. 2002/0012680 A1, published Jan. 31, 2002, the disclosure of which is herein incorporated by reference in its entirety. The following list therefore only offers a selection, which is by no means complete or exclusive, of several surfactant classes that are particularly common or useful in conjunction with present patent application. Preferred surfactants to be used in accordance with the disclosure include those with an HLB greater than 12. The list includes ionized long-chain fatty acids or long chain fatty alcohols, long chain fatty ammonium salts, such as alkyl- or alkenoyl-trimethyl-, -dimethyl- and -methyl-ammonium salts, alkyl- or alkenoyl-sulphate salts, long fatty chain dimethyl-aminoxides, such as alkyl- or alkenoyl-dimethyl-aminoxides, long fatty chain, for example alkanoyl, dimethyl-aminoxides and especially dodecyl dimethyl-aminoxide, long fatty chain, for example alkyl-N-methylglucamide-s and alkanoyl-N-methylglucamides. such as MEGA-8, MEGA-9 and MEGA-IO, N-long fatty chain-N,N-dimethylglycines, for example N-alkyl-N,N-dimethylglycines, 3-(long fatty chain-dimethylammonio)-alkane-sulphonates, for example 3-(acyidimethylammonio)-alkanesulphonatcs, long fatty chain derivatives of sulphosuccinate salts, such as bis(2-ethylalkyl) sulphosuccinate salts, long fatty chain-sulphobetaines, for example acyl-sulphobetaines, long fatty chain betaines, such as EMPIGEN BB or ZWITTERGENT-3-16, -3-14, -3-12, -3-10, or -3-8, or polyethylcn-glycol-acylphenyl ethers, especially nonaethylen-glycol-octyl-phenyl ether, polyethylene-long fatty chain-ethers, especially polyethylene-acyl ethers, such as nonaethylen-decyl ether, nonaethylen-dodecyl ether or octaethylene-dodecyl ether, polyethyleneglycol-isoacyl ethers, such as octaethyleneglycol-isotridecyl ether, polyethyleneglycol-sorbitane-long fatty chain esters, for example polyethyleneglycol-sorbitane-acyl esters and especially polyoxyethylene-monolaurate (e.g. polysorbate 20 or Tween 20), polyoxyethylene-sorbitan-monooleate (e.g. polysorbate 80 or Tween 80), polyoxyethylene-sorbitan-monolauroleylate, polyoxyethylene-sorbitan-monopetroselinate, polyoxyethylene-sorbitan-monoelaidate, polyoxyethylene-sorbitan-myristoleylate, polyoxyethylene-sorbitan-palmitoleinylate, polyoxyethylene-sorbitan-p-etroselinylate, polyhydroxyethylene-long fatty chain ethers, for example polyhydroxyethylene-acyl ethers, such as polyhydroxyethylene-lauryl ethers, polyhydroxyethylene-myristoyl ethers, polyhydroxyethylene-cetylst-earyl, polyhyd roxyethylene-palmityl ethers, polyhydroxyethylene-oleoyl ethers, polyhydroxyethylene-palmitoleoyl ethers, polyhydroxyethylene-lino-leyl, polyhydroxyethylen-4, or 6, or 8, or 10, or 12-lauryl, miristoyl, palmitoyl, palmitoleyl, oleoyl or linoeyl ethers (Brij series), or in the corresponding esters, polyhydroxyethylen-laurate, -myristate, -palmitate, -stearate or -oleate, especially polyhydroxyethylen-8-stearate (Myrj 45) and polyhydroxyethylen-8-oleate, polyethoxylated castor oil 40 (Cremophor EL), sorbitane-mono long fatty chain, for example alkylate (Arlacel or Span series), especially as sorbitane-monolaurate (Arlacel 20, Span 20), long fatty chain, for example acyl-N-methylglucamides, alkanoyl-N-methylglucamides, especially decanoyl-N-methylglucamide, dodecanoyl-N-methylglucamide, long fatty chain sulphates, for example alkyl-sulphates, alkyl sulphate salts, such as lauryl-sulphate (SDS), oleoyl-sulphate: long fatty chain thioglucosides, such as alkylthioglucosides and especially heptyl-, octyl- and nonyl-beta-D-thioglucopyranoside; long fatty chain derivatives of various carbohydrates, such as pentoses, hcxoses and disaccharidcs, especially alkyl-glucosides and maltosides, such as hexyl-, heptyl-, octyl-, nonyl- and decyl-beta-D-glucopyranoside or D-maltopyranosidc; further a salt, especially a sodium salt, of cholate, deoxycholate, glycocholate, glycodcoxycholate, taurodeoxycholate, taurocholate, a fatty acid salt, especially oleate, elaidate, linoleate, laurate, or myristate, most often in sodium form, lysophospholipids, n-octadecylene-glycerophosphatidic acid, octadecylene-phosphorylglycerol, octadecylene-phosphorylserine, n-long fatty chain-glycero-phosphatidic acids, such as n-acyl-glycero-phosphatidic acids, especially lauryl glycero-phosphatidic acids, oleoyl-glycero-phosphatidic acid, n-long fatty chain-phosphoryl glycerol, such as n-acyl-phosphorylglycerol, especially lauryl-, myristoyl-, oleoyl- or palmitoeloyl-phosphorylglycerol, n-long fatty chain-phosphorylserine, such as n-acyl-phosphoryl serine, especially lauryl-, myristoyl-, oleoyl- or palmitoeloyl-phosphorylserine, n-tetradecyl-glycero-phosphatidic acid, n-tetradecyl-phosphorylglycerol, n-tetradecyl-phosphoryl serine, corresponding-, elaidoyl-, vaccenyl-lysophospholipids, corresponding short-chain phospholipids, as well as all surface active and thus membrane destabilising polypeptides. Surfactant chains are typically chosen to be in a fluid state or at least to be compatible with the maintenance of fluid-chain state in carrier aggregates.

The surfactant may be a nonionic surfactant. The surfactant may be present in the formulation in about 0.2 to 10%, about 1% to about 10%, about 1% to about 7% or about 2% to 5% by weight. The nonionic surfactant may be selected from the group consisting of: polyoxyethylene sorbitans (polysobate surfactants), polyhydroxyethylene stearates or polyhydroxyethylene laurylethers (Brij surfactants). The surfactant may be a polyoxyethylene-sorbitan-monooleate (e.g. polysorbate 80 or Tween 80) or Tween 20, 40 or 60. The polysorbate may have any chain with 12 to 20 carbon atoms. The polysorbate may be fluid in the formulation, which may contain one or more double bonds, branching, or cyclo-groups.

The surfactant may be modified with additional PEG molecules or other hydrophilic moieties.

The formulations of the invention may comprise only one lipid and only one surfactant in addition to the modified lipid or surfactant. Alternatively, the formulations of the invention may comprise more than one lipid and only one surfactant, e.g., two, three, four, or more lipids and one surfactant. Alternatively, the formulations of the invention may comprise only one lipid and more than one surfactant, e.g., two, three, four, or more surfactants and one lipid. The formulations of the invention may comprise more than one lipid and more than one surfactant, e.g., two, three, four, or more lipids and two, three, four, or more surfactants.

The formulations of the invention may have a range of lipid to surfactant ratios (inclusive of the lipid and/or surfactant that is bonded to the AOI). The ratios may be expressed in terms of molar terms (mol lipid/mol surfactant). The molar ratio of lipid to surfactant in the formulations may be from about 1:3 to about 30:1, from about 1:2 to about 30:1, from about 1:1 to about 30:1, from about 2:1 to about 20:1, from about 5:1 to about 30:1, from about 10:1 to about 30:1, from about 15:1 to about 30:1, or from about 20:1 to about 30:1. The molar ratio of lipid to surfactant in the formulations of the invention may be from about 1:2 to about 10:1. The ratio may be from about 1:1 to about 2:1, from about 2:1 to about 3:1, from about 3:1 to about 4:1. from about 4:1 to about 5:1 or from about 5:1 to about 10:1. The molar ratio may be from about 10.1 to about 30:1, from about 10:1 to about 20:1, from about 10:1 to about 25:1, and from about 20:1 to about 25:1. The lipid to surfactant ratio may be about 1.0:1.0, about 1.25:1.0, about 1.5/1.0, about 1.75/1.0, about 2.0/1.0, about 2.5/1.0, about 3.0/1.0 or about 4.0/1.0. The formulations of the invention may also have varying amounts of total amount of the following components: lipid and surfactant combined (TA). The TA amount may be stated in terms of weight percent of the total composition. The TA may be from about 1% to about 40%, about 5% to about 30%, about 7.5% to about 15%, about 6% to about 14%, about 8% to about 12%, about 5% to about 10%, about 10% to about 20% or about 20% to about 30%. The TA may be 6%, 8%, 9%, 10%, 12%, 14%, 15% or 20%.

Selected ranges for total lipid amounts and lipid/surfactant ratios (mol/mol) for the formulations of the invention are described in the Table below:

TABLE 2
Total Amount and Lipid to Surfactant Ratios

| TA (and surfactant) (%) | Lipid/Surfactant (mol/mol) |
|---|---|
| 5 to 10 | 1.0 to 1.25 |
| 5 to 10 | 1.25 to 1.72 |
| 5 to 10 | 1.75 to 2.25 |
| 5 to 10 | 2.25 to 3.00 |
| 5 to 10 | 3.00 to 4.00 |
| 5 to 10 | 4.00 to 8.00 |
| 5 to 10 | 10.00 to 13.00 |
| 5 to 10 | 15.00 to 20.00 |
| 5 to 10 | 20.00 to 22.00 |
| 5 to 10 | 22.00 to 25.00 |
| 10 to 20 | 1.0 to 1.25 |
| 10 to 20 | 1.25 to 1.75 |
| 10 to 20 | 1.25 to 1.75 |
| 10 to 20 | 2.25 to 3.00 |
| 10 to 20 | 3.00 to 4.00 |
| 10 to 20 | 4.00 to 8.00 |
| 10 to 20 | 10.00 to 13.00 |
| 10 to 20 | 15.00 to 20.00 |
| 10 to 20 | 20.00 to 22.00 |
| 10 to 20 | 22.00 to 25.00 |

The formulations of the invention may optionally contain one or more of the following ingredients: co-solvents, chelators, buffers, antioxidants, preservatives, microbicides, emollients, humectants, lubricants and thickeners. Preferred amounts of optional components are described as follows.

| Antioxidant: | Molar (M) or | Rel w %* |
|---|---|---|
| Primary: | | |
| Butylated hydroxyanisole, BHA | | 0.1-8 |
| Butylated hydroxytoluene BHT | | 0.1-4 |
| Thymol | | 0.1-1 |
| Metabisulphite | 1-5 mM | |
| Bisulsphite | 1-5 mM | |
| Thiourea (MW = 76.12) | 1-10 mM | |
| Monothioglycerol (MW = 108.16) | 1-20 mM | |
| Propyl gallate (MW = 212.2) | | 0.02-0.2 |
| Ascorbate (MW = 175.3⁺ ion) | 1-10 mM | |
| Palmityl-ascorbate | | 0.01-1 |
| Tocopherol-PEG | | 0.5-5 |
| Secondary (chelator) | | |
| EDTA (MW = 292) | 1-10 mM | |
| EGTA (MW = 380.35) | 1-10 mM | |
| Desferal (MW = 656.79) | 0.1-5 mM | |
| Buffer | | |
| Acetate | 30-150 mM | |
| Phosphate | 10-50 mM | |
| Triethanolamine | 30-150 mM | |

*as a percentage of total lipid quantity

The formulations of the invention may include a buffer to adjust the pH of the aqueous solution to a range from pH 3.5 to pH 9, pH 4 to pH 7.5, or pH 6 to pH 7. Examples of buffers include, but are not limited to. acetate buffers, lactate buffers, phosphate buffers, and propionate buffers.

The formulations of the invention are typically formulated in aqueous media. The formulations may be formulated with or without co-solvents, such as lower alcohols. The formulations of the invention may comprise at least 20% by weight water. The formulations of the invention may comprise about 20%, about 30%, about 40%, about 50%, about 60% about 70%, about 80%, about 90% by weight water. The formulation may comprise from about 70% to about 80% by weight water.

A “microbicide” or “antimicrobial” agent is commonly added to reduce the bacterial count in pharmaceutical formulations. Some examples of microbicides are short chain alcohols, including ethyl and isopropyl alcohol, chlorbutanol, benzyl alcohol, chlorbenzyl alcohol, dichlorbenzylalcohol, hexachlorophene; phenolic compounds, such as cresol, 4-chloro-m-cresol, p-chloro-m-xylenol. dichlorophene, hexachlorophene, povidon-iodine; parabenes. especially alkyl-parabenes, such as methyl-, ethyl-, propyl-, or butyl-paraben, benzyl paraben; acids, such as sorbic acid, benzoic acid and their salts; quaternary ammonium compounds, such as alkonium salts, e.g., a bromide, benzalkonium salts, such as a chloride or a bromide, cetrimonium salts, e.g., a bromide, phenoalkecinium salts, such as phenododecinium bromide, cetylpyridinium chloride and other salts; furthermore, mercurial compounds, such as phenylmercuric acetate, borate, or nitrate, thiomersal, chlorhexidine or its gluconate, or any antibiotically active compounds of biological origin, or any suitable mixture thereof.

Examples of “antioxidants” are butylated hydroxyanisol (BHA), butylated hydroxytoluene (BHT) and di-tert-butylphenol (LY178002, LY256548, HWA-131, BF-389, CI-986, PD-127443, E-51 or 19, BI-L-239XX, etc.), tertiary butylhydroquinone (TBHQ), propyl gallate (PG), 1-O-hexyl-2,3,5-trimethylhydroquinone (HTHQ); aromatic amines (diphenylamine, p-alkylthio-o-anisidine, ethylenediamine derivatives, carbazol, tetrahydroindenoindol); phenols and phenolic acids (guaiacol, hydroquinone, vanillin, gallic acids and their esters, protocatechuic acid, quinic acid, syringic acid, ellagic acid, salicylic acid, nordihydroguaiaretic acid (NDGA), eugenol); tocopherols (including tocopherols (alpha, beta, gamma, delta) and their derivatives, such as tocopheryl-acylate (e g. -acetate. -laurate. myristate, -palmitate, -oleate, -linoleate. etc., or an y other suitable tocopheryl-lipoate). tocopheryl-POE-succinate; trolox and corresponding amide and thiocarboxamide analogues; ascorbic acid and its salts, isoascorbate, (2 or 3 or 6)-o-alkylascorbic acids, ascorbyl esters (e.g., 6-o-lauroyl, myristoyl, palmitoyl-, oleoyl, or linoleoyl-L-ascorbic acid, etc.). Also useful are the preferentially oxidised compounds, such as sodium bisulphite, sodium metabisulphite, thiourea; chellating agents, such as EDTA, GDTA, desferral: miscellaneous endogenous defence systems, such as transferrin, lactoferrin, ferritin, cearuloplasmin, haptoglobion, heamopexin, albumin, glucose, ubiquinol-10); enzymatic antioxidants, such as superoxide dismutase and metal complexes with a similar activity, including catalase, glutathione peroxidase, and less complex molecules, such as beta-carotene, bilirubin, uric acid; flavonoids (flavones, flavonols, flavonones, flavanonals, chacones, anthocyanins). N-acetylcystein, mesna. glutathione, thiohistidine derivatives, triazoles; tannines, cinnamic acid, hydroxycinnamatic acids and their esters (coumaric acids and esters, caffeic acid and their esters, ferulic acid, (iso-) chlorogenic acid, sinapic acid); spice extracts (e.g., from clove, cinnamon, sage, rosemary, mace, oregano, allspice, nutmeg); carnosic acid, carnosol, carsolic acid; rosmarinic acid, rosmaridiphenol, gentisic acid, ferulic acid; oat flour extracts, such as avenanthramide 1 and 2; thioethers, dithioethers, sulphoxides, tetralkylthiuram disulphides; phytic acid, steroid derivatives (e.g., U74006F); tryptophan metabolites (e.g., 3-hydroxykynurenine, 3-hydroxyanthranilic acid), and organochalcogenides.

“Thickeners” are used to increase the viscosity of pharmaceutical formulations to and may be selected from selected from pharmaceutically acceptable hydrophilic polymers, such as partially etherified cellulose derivatives, comprising carboxym ethyl-, hydroxyethyl-, hydroxypropyl-, hydroxypropylmethyl- or methyl-cellulose; completely synthetic hydrophilic polymers comprising polyacrylates, polymethacrylatcs, poly(hydroxyethyl)-, poly(hydroxypropyl)-, poly(hydroxypropylmethyl)methacrylate, polyacrylonitrile, methallyl-sulphonate, polyethylenes, polyoxiethylenes, polyethylene glycols, polyethylene glycol-lactide, polyethylene glycol-diacrylate, polyvinylpyrrolidone, polyvinyl alcohols, poly(propylmethacrylamide), poly(propylene fumarate-co-ethylene glycol), poloxamers, polyaspartamide. (hydrazine cross-linked) hyaluronic acid, silicone; natural gums comprising alginates, carrageenan, guar-gum, gelatine, tragacanth, (amidated) pectin, xanthan, chitosan collagen, agarose; mixtures and further derivatives or co-polymers thereof and/or other pharmaceutically, or at least biologically, acceptable polymers.

The formulations of the present invention may also comprise a polar liquid medium. The formulations of the invention may be administered in an aqueous medium. The formulations of the present invention may be in the form of a solution, suspension, emulsion, cream, lotion, ointment, gel, spray, film forming solution or lacquer.

While not to be limited to any mechanism of action or any theory, the formulations of the invention may form vesicles or ESAs characterized by their adaptability, deformability, or penetrability. Similar vesicles (without a therapeutic entity bonded) are described in both WO 2010/140061 and in WO 2011/022707.

The formulations of the invention are useful in the prevention or treatment of a variety of diseases or conditions, depending on the AOI, as mentioned above.

For example, the vesicular formulations of the invention may comprise one, two or three of vitamins D₃, C, E or B₇. The formulation may be used alone or as a component or ingredient of a more complex skin care product such as a sunscreen, sun block, moisturiser, serum, or cosmetics. The formulation or final skin care product may be in the form of a cream, gel, lotion, mousse or spray.

Provided by the invention is a vesicular formulation for use as defined above, wherein the micronutrient is vitamin D₃ the formulation may be incorporated into a sunscreen product, a sun block product, an after-sun product or other skincare or cosmetic product to supplement low vitamin D levels; wherein the micronutrient is vitamin C or vitamin E, the formulation may be incorporated into a sunscreen product, a sun block product, an after-sun product or other skincare or cosmetic product to supplement epidermal and dermal vitamin C or vitamin E and assist in the prevention or repair of sun-damaged or aging skin; wherein the micronutrient is vitamin B₇ the formulation may be incorporated into a sunscreen product, a sun block product, an after-sun product or other skincare or cosmetic product to reduce or eliminate dermatoses associated with a lack of this micronutrient.

The vesicular formulation of the invention may be provided in a wound-healing product to be applied topically. Thus, the present invention provides the formulation of the first aspect for use in treating a wound of the skin, wherein the AOI is ascorbic acid (vitamin C).

The invention is described below with reference to the following non-limiting examples and figures, in which:

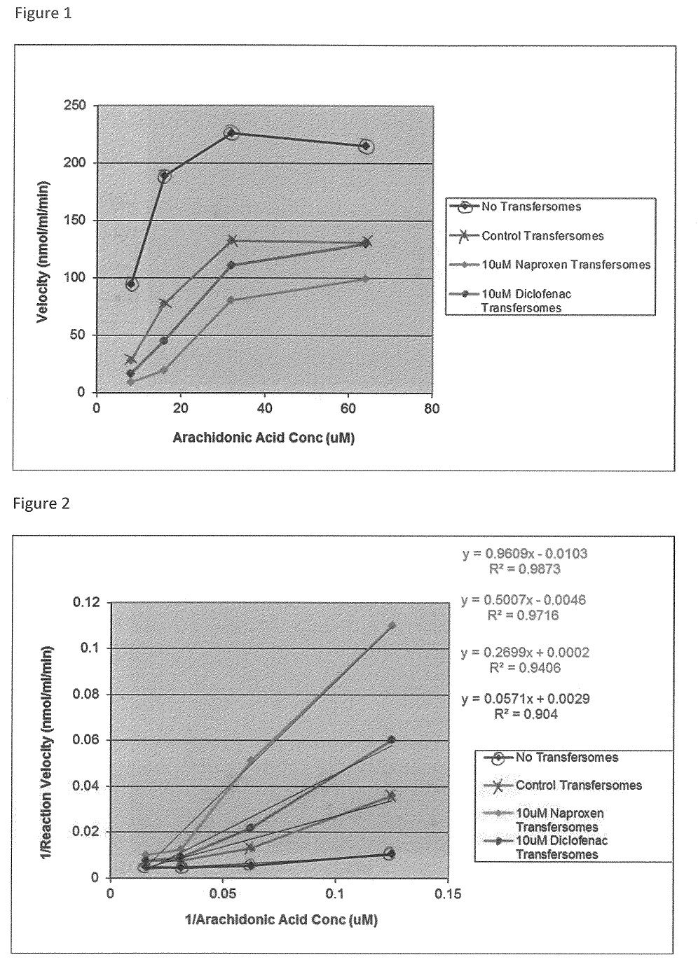

FIG. 1 shows the arachidonic substrate concentration plotted against the velocity of reaction for the vesicles tethered to Naproxen or Diclofenac;

FIG. 2 shows the reciprocal (Lineweaver Burk) plot of FIG. 1;

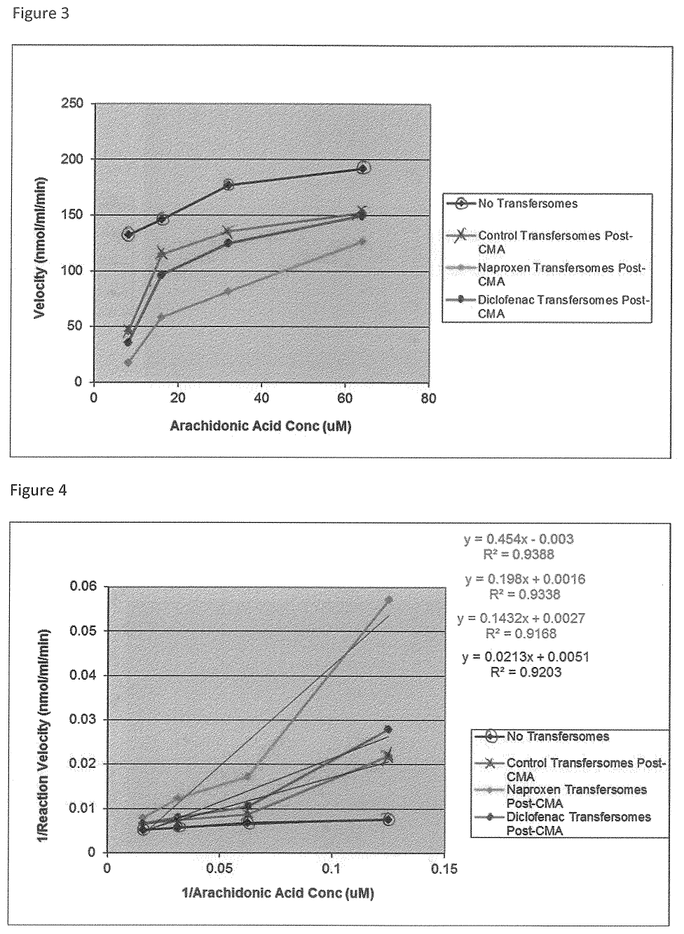

FIG. 3 shows the arachidonic substrate concentration plotted against the velocity of reaction for vesicles tethered to Naproxen or Diclofenac after a CMA assy; and

FIG. 4 shows the reciprocal (Lineweaver Burk) plot of FIG. 3.

### EXAMPLE FORMULATIONS

### Example Vesicular Formulations

### Example Formulation 1

Formulation 1 comprises sphingomyelin (brain) (47.944 mg/g) as a lipid, Tween 80 (42.05 mg/g) as a surfactant, lactate buffer (pH 4). benzyl alcohol or paraben (5.000 mg/g) as an antimicrobial agent, BHT (0.200 mg/g) and sodium metabisulfite (0.0500 mg/g) as antioxidants, glycerol (30.000 mg/g), EDTA (3.000 mg/g) as a chelating agent, and ethanol (30.000 mg/g).

### Example Formulation 2

Formulation 2 comprises sphingomyelin (brain) (53.750 mg/g) as a lipid, Tween 80 (31.250 mg/g) as a surfactant, lactate (pH 4) buffer, benzyl alcohol or paraben (5.000 mg/g) as an antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (15.000 mg/g).

### Example Formulation 3

Formulation 3 comprises sphingomyelin (brain) (90.561 mg/g) as a lipid, Tween 80 (79.439 mg/g) as a surfactant, lactate (pH 4) buffer, benzyl alcohol or paraben (5.000 mg/g) as an antimicrobial agent, BHT (0.200 mg/g) and sodium metabisulfite (0.500 mg/g) as antioxidants, glycerol (30.000 mg/g), EDTA (3.000 mg/g) as a chelating agent, and ethanol (30.000 mg/g).

### Example Formulation 4

Formulation 4 comprises phosphatidyl choline (68.700 mg/g) as a lipid, Tween 80 (8.500 mg/g) as a surfactant, phosphate (pH 7.5) buffer, BHT (0.200 mg/g) and sodium metabisulfite (0.500 mg/g) as antioxidants, benzyl alcohol or paraben (5.000 mg/g) as an antimicrobial, glycerol (30.000 mg/g), EDTA (1.000 mg/g) as a chelating agent, and ethanol (36.51 mg/g).

### Example Formulation 5

Formulation 5 comprises phosphatidyl choline (71.460 mg/g) as a lipid, Tween 80 (4.720 mg/g) as a surfactant, phosphate (pH 7.8) buffer. BHA (0.200 mg/g) and sodium metabisulfite (0.500 mg/g) as antioxidants, benzyl alcohol or paraben (5.000 mg/g) as an antimicrobial, glycerol (15.000 mg/g), EDTA (3.000 mg/g) as a chelating agent, and ethanol (35.000 mg/g).

### Example Formulation 6

Formulation 6 comprises phosphatidyl choline (71.460 mg/g) as a lipid, Tween 80 (4.720 mg/g) as a surfactant, phosphate (pH 7.8) buffer, BHA (0.200 mg/g) and sodium metabisulfite (0.500 mg/g) as antioxidants, glycerol (50.000 mg/g), EDTA (3.000 mg/g) as a chelating agent, and ethanol (15.000 mg/g).

### Example Formulation 7

Formulation 8 comprises phosphatidyl choline (71.4600 mg/g) as a lipid, Tween 80 (4.720 mg/g) as a surfactant, phosphate (pH 7.5) buffer, BHA (0.200 mg/g) and sodium metabisulfite (0.500 mg/g) as antioxidants, glycerol (50.000 mg/g), EDTA (3.000 mg/g) as a chelating agent, and ethanol (35.000 mg/g).

### Example Formulation 8

Formulation 8 comprises phosphatidyl choline (64.516 mg/g) as a lipid, Tween 80 (35.484 mg/g) as a surfactant, phosphate (pH 6.7) buffer, BHA (0.200 mg/g) as antioxidant, benzyl alcohol or paraben (4.200 mg/g) as an antimicrobial, glycerol (30.000 mg/g), EDTA (3.000 mg/g) as a chelating agent, and ethanol (30.000 mg/g).

### Example Formulation 9

Phosphatidylcholine (64.516 mg/g) as a lipid, Tween 80 (35.484 mg/g) as a surfactant, phosphate (pH 6.7) buffer, BHA (0.200 mg/g) as an antioxidant, benzyl alcohol (5.250 mg/g) or paraben (4.200 mg/g) as a solvent, glycerol (30.000 mg/g), EDTA (3.000 mg/g) as a chelating agent, and ethanol (30.000 mg/g).

### Example Formulation 10

Phosphatidyl choline (71.460 mg/g) as a lipid, Tween 80 (4.720 mg/g) as a surfactant, phosphate (pH 6.7) buffer, BHA (0.200 mg/g) as antioxidant, benzyl alcohol or paraben (10.000 mg/g) as a solvent, glycerol (50.000 mg/g), EDTA (3.000 mg/g) as a chelating agent, and ethanol (30.000 mg/g).

### Example Vesicular Formulations with an AOI Attached

### Example Formulation 11

Formulation 9 comprises phosphatidyl choline (68.700 mg/g) as a lipid, Tween 80 (8.500 mg/g) as a surfactant, collagenyl phosphatidylcholine (1 mg/g) as a AOI, phosphate (pH 7.5) buffer, BHT (0.200 mg/g) and sodium metabisulfite (0.500 mg/g) as antioxidants, benzyl alcohol or paraben (5.000 mg/g) as an antimicrobial, glycerol (30.000 mg/g), EDTA (1.000 mg/g) as a chelating agent, and ethanol (36.51 mg/g).

### Example Formulation 12

Formulation 10 comprises phosphatidyl choline (68.700 mg/g) as a lipid, Tween 80 (8.500 mg/g) as a surfactant, collagenyl phosphatidylcholine (0.5 mg/g) as a AOI, phosphate (pH 7.5) buffer, BHT (0.200 mg/g) and sodium metabisulfite (0.500 mg/g) as antioxidants, benzyl alcohol or paraben (5.000 mg/g) as an antimicrobial, glycerol (30.000 mg/g), EDTA (1.000 mg/g) as a chelating agent, and ethanol (36.51 mg/g).

### Example Formulation 13

Formulation 11 comprises phosphatidyl choline (68.700 mg/g) as a lipid, Tween 80 (8.500 mg/g) as a surfactant, collagenyl Tween (0.5 mg/g), phosphate (pH 7.5) buffer, BHT (0.200 mg/g) and sodium metabisulfite (0.500 mg/g) as antioxidants, benzyl alcohol or paraben (5.000 mg/g) as an antimicrobial, glycerol (30.000 mg/g), EDTA (1.000 mg/g) as a chelating agent, and ethanol (36.51 mg/g).

### Example 14 and Manufacture and Testing Thereof

Formulation 14 comprises phosphatidyl choline (68.700 mg/g) as a lipid, Tween 80 (6.800 mg/g) as a surfactant, ascorbyl palmitate (0.530 mg/g) as an AOI, citrate phosphate (pH 5.4) buffer, BHT (0.200 mg/g) and sodium metabisulfite (0.500 mg/g) as antioxidants, EDTA (1.000 mg/g) as a chelating agent, and ethanol (48.87 mg/g).

### SUMMARY

A Transfersome preparation has been successfully manufactured to contain covalently bonded ascorbic acid at 20% polysorbate 80 molar substitution. Test results showed that the size distribution, deformability characteristics and charge of the transfersomes were unaffected by the inclusion of the ascorbic acid, that the L-ascorbyl palmitate ester was accessible on the external surface of the transfersome to a carboxylesterase enzyme, that the ascorbyl palmitate transfersomes were active in an Fe³⁺ reducing assay and that they retained their reducing activity after deforming to pass through pores that were smaller than their average size.

### Manufacture

Transfersomes were prepared using soybean phosphatidylcholine (Lipoid SPC S-100) and polysorbate 80, containing L-ascorbyl palmitate (Sigma 7618). A control batch of transfersomes was also made. Butylhydroxytoluene, EDTA and sodium metabisulphite were added to the transfersomes to minimize oxidation of L-ascorbyl palmitate.

### Preparation of L-Ascorbyl Palmitate Transfersomes

A 50 g batch of L-ascorbyl palmitate transfersomes was prepared with soybean phosphatidylcholine: polysorbate 80: L-ascorbyl palmitate molar ratios of 13.3:0.8:0.2

Using gentle heat and stirring, soybean phosphatidylcholine (3.44 g), polysorbate 80 (0.34 g), butylhydroxytoluene (0.01 g) and L-ascorbyl palmitate (0.0265 g) were dissolved in ethanol to give a total weight of 6.26 g.

25 mM citrate phosphate buffer pH5.4, with 0.1% EDTA and 0.05% sodium metabisulphite, (43.74 g) was stirred vigorously at 35° C. while the soybean phosphatidylcholine preparation was added from a syringe fitted with a wide gauge needle. The mixture was stirred for approximately 15 minutes.

The transfersomes were prepared by extrusion through a 0.2 μm filter, followed by a 0.1 μm filter and a further 0.1 μm filter using a Sartorius 47 mm filter system at 35° C. with nitrogen at 4 bar pressure. Each filter had a glass fibre pre-filter on top. Transfersomes were stored in the dark at +5° C.

### Preparation of Control Transfersomes

A 50 g batch of control transfersomes was prepared with a soybean phosphatidylcholine: polysorbate 80 molar ratio of 13.3:1

Using gentle heat and stirring, soybean phosphatidylcholine (3.44 g), polysorbate 80 (0.425 g) and butylhydroxytoluene (0.01 g) were dissolved in ethanol to give a total weight of 6.26 g.

25 mM citrate phosphate buffer pH5.4, with 0.1% EDTA and 0.05% sodium metabisulphite, (43.74 g) was stirred vigorously at 35° C. while the soybean phosphatidylcholine preparation was added from a syringe fitted with a wide gauge needle. The mixture was stirred for approximately 15 minutes.

The control transfersomes were extruded as described for L-ascorbyl palmitate transfersome batch PD-14-0035. Transfersomes were stored in the dark at +5° C.

### Analytical Methods

### Particle Size Measurement

The average particle size and the particle size distribution for the transfersome preparations were determined by dynamic light scattering using a photon correlation spectrometer. When coherent light is passed through a suspension of particles, light is scattered in all directions. By measurement and correlation of the scattered light intensity of a particle suspension, it is possible to determine the size and size distribution of the particles in the suspension.

The mean particle size and particle size distribution for each sample were determined using an ALV-5000/E photon correlation spectrometer. Samples were diluted in de-ionised water to give a detectable signal within the range of 50-500 kHz, and then analysed over six measurements, each of 30 seconds duration. The temperature was controlled at 25° C. The data was subjected to a regularised fit cumulative second order analysis to give the mean particle size (reported as r or the mean radius) as well as the particle sizing distribution for the sample (reported as w or width). The mean radius was multiplied by 2 to give the mean diameter (nm).

The polydispersity index (PDI) for each sample was calculated according to the following equation:

$$PDI = {( \frac{w}{r} )}^{2}$$

where: w=width and r=average radius.

### Continuous Membrane Adaptability Assay

The continuous membrane adaptability (CMA) assay used applied pressure to provide activation energy to transfersomes to enable them to deform and pass through a filter pore that is smaller than the average size of the transfersomes.

An Anodisc 13 membrane filter (pore size 20 nm) was mounted on a filtration support in the base of a filtering device and the upper stainless steel barrel was attached. 3 ml transfersome sample pre-equilibrated at 25° C. was placed in the barrel and heat transmitting tube connected to a thermocirculator (25° C.) was wrapped around it. The barrel was connected to a pressure tube connected to a Nitrogen cylinder. Using a series of valves, the system was primed with set-point of 9.5 bar pressure to give 7.5 bar starting pressure. The filtration device was placed over a collection vessel sited on a precision weighing balance that was connected to an Excel computer program. A Bronkhurst pressure controller was used to control and monitor the pressure and when the system valves were opened and timing started, the increasing mass of transfersome filtrate collected on the balance was recorded against the decreasing pressure and increasing time.

The time, pressure, mass data was evaluated in a MathCAD program to determine a P* value. P* is a measure of the activation pressure required for pore penetration and therefore a measure of transfersome membrane stiffness and deformability. The average particle size of the transfersomes was measured by photon correlation spectroscopy before and after the CMA filtration.

### Ascorbic Acid Assay

Ascorbyl palmitate and ascorbic acid concentrations were measured using an Ascorbic Acid Assay Kit (Abcam ab65656). In this assay, Fe³⁺ is reduced to Fe²⁺ in the presence of antioxidants such as ascorbic acid. The Fe²⁺ is chelated with a colorimetric probe to produce a product with absorbance at 593 nm.

To determine the total ascorbyl palmitate concentration, transfersomes were solubilised by dilution 1:7:2 v/v with ethanol and 5% Triton X-100. An ascorbyl palmitate standard curve was prepared by initial dilution in ethanol to a concentration range 0.0125 to 0.25 mM, then further diluted 7:1:2 v/v in water and 5% Triton X-100 to a final concentration range of 0.01 to 0.175 mM. A 0 mM ascorbyl palmitate blank was included. Standards and samples were loaded onto a microtitre plate and mixed 1:1 v/v with a reaction mixture containing kit buffer, Fe′ and colorimetric probe. After 1 minute incubation at room temperature, the plate was read at 593 nm. The 0 mM ascorbyl palmitate blank was subtracted from all standards and samples and the absorbance for control ‘empty’ transfersomes was subtracted from that of the ascorbyl palmitate transfersomes. The final absorbance was compared against the ascorbyl palmitate standard curve to obtain the total ascorbyl palmitate (ascorbic acid) concentration (mM).

To determine the external ascorbic acid concentration, an ascorbic acid standard curve was prepared by diluting ascorbic acid in water to a concentration range of 0.025 to 0.2 mM. Standards and transfersome samples were loaded onto a microtitre plate and mixed 1:1 v/v with reaction mixture containing kit buffer, Fe³⁺ and colorimetric probe. After 1 minute incubation at room temperature, the plate was read at 593 nm. The plate blank was subtracted from all standards and samples and the absorbance for control ‘empty’ transfersomes was subtracted from that of the ascorbyl palmitate transfersomes. The final absorbance was compared against the ascorbic acid standard curve to obtain ascorbic acid concentration. The concentration was compared with the total ascorbyl palmitate (ascorbic acid) concentration to calculate the % ascorbic acid tethered on the external surface of the ascorbyl palmitate transfersomes.

### Carboxylesterase Digest and Rp-HPLC

Release of ascorbic acid from transfersomes containing ascorbyl palmitate was performed by enzymatic digestion of the ester using Carboxylesterase 1 isoform B (Sigma E0287). 960 units of enzyme were added per ml of transfersomes, before incubation at +37° C. Samples were taken at 2 and 4 hours and the released ascorbic acid extracted by adding 1 volume of acetonitrile/methanol/formic acid (80 v/20 v/0.2 v) followed by sonication for 5 minutes and centrifugation to pellet insoluble components. Supernatant samples were then filtered through a 0.2 μm membrane before diluting 1 in 10 with ultra-high purity water.

Samples were assayed by a reversed phase high pressure liquid chromatography (RP-HPLC) method using a Luna C18(2) 100 A 5 μm 4.6×250 mm column and Waters 2695 separation module at +25° C. and a gradient method as per the table below where eluent A was 20 mM potassium phosphate pH3.0 and eluent B was acetonitrile. Detection was performed at a wavelength of 260 nm using a Waters 2487 detector.

| Time | Flow | | |
|---|---|---|---|
| (minutes) | (ml/min) | % Eluent A | % Eluent B |
| 0 | 1 | 95 | 5 |
| 10 | 1 | 87 | 13 |
| 11 | 1 | 35 | 65 |
| 15 | 1 | 35 | 65 |
| 16 | 1 | 95 | 5 |
| 25 | 1 | 95 | 5 |

In addition, a standard curve of ascorbic acid in the range of 0.4 to 100 μg/ml was analysed using the same RP-HPLC method. The ascorbic acid peak was integrated in the resulting chromatograms for the samples and standards. The peak areas of the standards were analysed with linear regression to produce an equation for the standard curve. The peak areas for the samples were then used to determine the ascorbic acid concentration from the equation for the standard curve taking into account the dilution from the extraction method. The concentration was compared with the total ascorbic acid concentration to calculate the % ascorbic acid released from the external surface of the ascorbyl palmitate transfersomes.

### Paper Electrophoresis

The charge characteristics of transfersome preparations were investigated using paper electrophoresis where a paper strip was suspended between two buffer filled reservoirs, the test sample was applied to the strip and an electrical current applied across the strip. Charged particles migrated across the strip, with the direction and distance travelled being determined by the net charge of the particles at the buffer pH.

Volumes (100 μl) of each test sample were applied to the centre of individual 2×20 cm Whatman filter strips (pre-wetted in running buffer; 2.3 mg/ml sodium chloride, 1.5 mg/ml calcium chloride, 1.3 mg/ml glycyl glycine, 25 mg/ml mannitol, 10 mg/ml sucrose and 0.5 mg/ml methionine at pH5.75) and run at 130V for 2 hours. Each strip was stained for PEG (a component of polysorbate 80) with 5% w/v barium chloride and 0.05M iodine and then dried. The extent of travel of the transfersomes away from the centre point for each sample was measured for both the anode and cathode sides of the strip to determine the vesicle net charge.

### Results and Discussion

Results are summarised in Table 4. Samples of the ascorbyl palmitate transfersomes were 0.2 μm sterile filtered and retested post filtration in order to recheck the integrity of the samples for information.

TABLE 4
Control and Ascorbyl Palmitate Transfersomes Analysis

| | | | Ascorbyl | Ascorbyl |
|---|---|---|---|---|
| | | | Palmitate | Palmitate |
| | | Ascorbyl | Trans- | Trans- |
| | Control | Palmitate | fersomes | fersomes |
| | Trans- | Trans- | Post 0.2 um | Post CMA |
| Test | fersomes | fersomes | Filtration | Assay |
| Photon | | | | |
| Correlation | | | | |
| Spectrometry: | | | | |
| Average Particle | 138.64 | 141.32 | 140.90 | 74.26 |
| Diameter (nm) | | | | |
| Polydispersity | 0.050 | 0.063 | 0.062 | 0.127 |
| Index | | | | |
| CMA Assay: | | | | |
| Filtration | 20.3 | 13.3 | N/A | N/A |
| Recovery % | | | | |
| Deformability P* | 1.622 | 1.698 | N/A | N/A |
| Ascorbic Acid | | | | |
| Assay: | | | | |
| Total Ascorbyl | N/A | 0.61 mM/ | 0.67 mM/ | 0.79 mM/ |
| Palmitate | | 253 μg/ml | 278 μg/ml | 327 μg/ml |
| Concentration | | | | |
| Total Ascorbic | N/A | 0.61 mM/ | 0.67 mM/ | 0.79 mM/ |
| Acid (AA) | | 107 μg/ml | 118 μg/ml | 139 μg/ml |
| Concentration | | | | |
| External AA | N/A | 0.13 mM/ | 0.12 mM/ | 0.14 mM/ |
| Concentration | | 23 μg/ml | 21 μg/ml | 25 μg/ml |
| % External AA | N/A | 21% | 18% | 18% |
| Carboxylesterase | | | | |
| Digest/HPLC: | | | | |
| Released External | N/A | 16 μg/ | N/A | 13 μg/ |
| AA Concentration | | ml (15%) | | ml (9%) |
| (2 hours 37° C.) | | | | |
| Released External | N/A | 38 μg/ | N/A | 26 μg/ |
| AA Concentration | | ml (36%) | | ml (19%) |
| (4 hours 37° C.) | | | | |
| Paper | Net positive | Net positive | N/A | N/A |
| Electrophoresis | charge | charge | | |

### Particle Size Measurement

The average particle diameter and polydispersity index were similar for the control transfersomes and for those containing ascorbyl palmitate. This indicated that 20% substitution of polysorbate 80 with the ester in the transfersomes had not affected the size characteristics. There was no significant change in size post 0.2 μm sterile filtration.

### Continuous Membrane Adaptability Assay

The deformability P* value was virtually the same for the transfersomes containing the ascorbyl palmitate and the control transfersomes, indicating that 20% substitution of polysorbate 80 with the ester had not significantly affected the deformability properties of the transfersomes. The filtration % recovery was slightly higher for the control transfersomes which could indicate that the inclusion of ascorbyl palmitate had a very slight stiffening effect on the vesicle membrane.

The average particle diameter post-CMA filtration decreased by almost 50% compared with pre-filtration for both the control transfersomes and for the transfersomes containing the ascorbyl palmitate ester. The polydispersity index was slightly higher, indicating a broader size distribution. These characteristics are as expected for transfersome vesicles.

### Ascorbic Acid Assay

The total ascorbyl palmitate concentration in ascorbyl palmitate transfersomes was determined as 0.61 mM. This equates to 253 μg/ml ascorbyl palmitate or 107 μg/ml ascorbic acid. The concentration was approximately 50% of that at the start of the manufacturing process, indicating that losses had occurred, probably through a combination of filtration and ascorbic acid oxidisation. However, results showed that active ascorbic acid capable of reducing Fe³⁺ was present in the final transfersome preparation.

The concentration of ascorbic acid that reacted on the external surface of the ascorbyl palmitate transfersomes was determined as 0.13 mM. This equates to 23 μg/ml or 21% of the total ascorbic acid concentration being externally tethered.

There was no significant change in total or external ascorbic acid concentration post 0.2 μm sterile filtration.

The total ascorbyl palmitate concentration of transfersomes that had been subjected to the continuous membrane adaptability (CMA) assay was slightly higher than pre-CMA. The external ascorbic acid concentration was virtually the same pre/post-CMA. This showed that transfersomes that had deformed to pass through a pore size that was smaller than their average diameter did not lose any of their reducing activity.

### Carboxylesterase Digest and Rp-HPLC

Incubation of transfersomes containing ascorbyl palmitate ester with carboxylesterase 1 enzyme resulted in the release of 15% (16 μg/ml) of the total ascorbic acid after 2 hours incubation at 37° C. and 36% (38 μg/ml) after 4 hours at 37° C. Ascorbic acid that was tethered to the external surface of the transfersome was therefore accessible to the enzyme. The concentrations obtained for external ascorbic acid were similar to those obtained in the ascorbic acid assay.

Transfersomes that had been subjected to the CMA deformability filtration assay were also incubated with carboxylesterase 1 enzyme resulting in the release of 9% (13 μg/ml) of the total ascorbic acid after 2 hours incubation at 37° C. and 19% (26 μg/ml) after 4 hours at 37° C. It is unclear why the percentage release was lower post CMA, but possibly the change in vesicle size reduced the accessibility of the ascorbyl palmitate to the enzyme.

### Paper Electrophoresis

Control transfersomes and transfersomes containing ascorbyl palmitate both migrated towards the cathode of the electrophoresis apparatus, demonstrating a net positive charge. The presence of ascorbyl palmitate did not therefore alter the charge characteristics of the transfersomes.

### Example Formulation 15 and Manufacture and Testing Thereof

Formulation 15 comprises either phosphatidyl choline (68.700 mg/g) as a lipid, Tween 80 (7.66 mg/g) as a surfactant, palmitoyl tripeptide 1 (0.370 mg/g) as an AOI, phosphate (pH 7.7) buffer and ethanol (48.10 mg/g), or phosphatidyl choline (68.700 mg/g) as a lipid, Tween 80 (7.66 mg/g) as a surfactant, palmitoyl tetrapeptide 7 (0.450 mg/g) as an AOI, phosphate (pH 7.7) buffer and ethanol (48.40 mg/g).

### SUMMARY

Transfersome preparations have successfully been manufactured to contain covalently bonded peptides; tetrapeptide-7 and tripeptide-1; at 10% polysorbate 80 molar substitution. Test results showed that the size distribution, deformability characteristics and charge of the transfersomes were unaffected by the inclusion of the peptides.

### Manufacture

Transfersomes were prepared using soybean phosphatidylcholine (Lipoid SPC S-100) and polysorbate 80 containing either palmitoyl tetrapeptide-7 (PAL-GQPR) or palmitoyl tripeptide-1 (PAL-GHK) (Sinoway Industrial Co. Ltd). A control batch of transfersomes was also made.

### Preparation of Palmitoyl Peptide Transfersomes

A 50 g batch of palmitoyl tetrapeptide-7 transfersomes and a 50 g batch of palmitoyl tripeptide-1 transfersomes were prepared with soybean phosphatidylcholine: polysorbate 80: palmitoyl peptide molar ratios of 13.3:0.9:0.1

Using gentle heat and stirring, soybean phosphatidylcholine (3.44 g), polysorbate 80 (0.383 g) and EITHER palmitoyl tetrapeptide-7 (0.0224 g) palmitoyl tripeptide-1 (0.0186 g) were dissolved in ethanol to give a total weight of 6.26 g.

Phosphate buffer, pH7.7 (43.74 g) was stirred vigorously at 35° C. while the soybean phosphatidylcholine preparation was added from a syringe fitted with a wide gauge needle. The mixture was stirred for approximately 15 minutes.

The transfersomes were prepared by extrusion through a 0.2 μm filter, followed by a 0.1 μm filter and a further 0.1 μm filter using a Sartorius 47 mm filter system at 35° C. with nitrogen at 4 bar pressure. Each filter had a glass fibre pre-filter on top. Transfersomes were stored in the dark at +5° C.

### Preparation of Control Transfersomes

A 50 g batch of control transfersomes was prepared with a soybean phosphatidylcholine: polysorbate 80 molar ratio of 13.3:1.

Using gentle heat and stirring, soybean phosphatidylcholine (3.44 g) and polysorbate 80 (0.425 g) were dissolved in ethanol to give a total weight of 6.26 g.

Phosphate buffer, pH7.7 (43.74 g) was stirred vigorously at 35° C. while the soybean phosphatidylcholine preparation was added from a syringe fitted with a wide gauge needle. The mixture was stirred for approximately 15 minutes.

The control transfersomes were extruded as described for palmitoyl peptide transfersomes batches. Transfersomes were stored in the dark at +5° C.

### Analytical Methods

### Particle Size Measurement

The average particle size and the particle size distribution for transfersome preparations were determined by dynamic light scattering using a photon correlation spectrometer. When coherent light is passed through a suspension of particles, light is scattered in all directions. By measurement and correlation of the scattered light intensity of a particle suspension, it is possible to determine the size and size distribution of the particles in the suspension.

The mean particle size and particle size distribution for each sample were determined using an ALV-5000/E photon correlation spectrometer. Samples were diluted in de-ionised water to give a detectable signal within the range of 50-500 kHz, and then analysed over six measurements, each of 30 seconds duration. The temperature was controlled at 25° C. The data was subjected to a regularised fit cumulative second order analysis to give the mean particle size (reported as r or the mean radius) as well as the particle sizing distribution for the sample (reported as w or width). The mean radius was multiplied by 2 to give the mean diameter (nm).

The polydispersity index (PDI) for each sample was calculated according to the following equation:

$$PDI = {( \frac{w}{r} )}^{2}$$

where: w=width and r=average radius.

### Continuous Membrane Adaptability Assay

The continuous membrane adaptability (CMA) assay used applied pressure to provide activation energy to transfersomes to enable them to deform and pass through a filter pore that is smaller than the average size of the transfersomes.

An Anodisc 13 membrane filter (pore size 20 nm) was mounted on a filtration support in the base of a filtering device and the upper stainless steel barrel was attached. 3 ml of transfersome sample pre-equilibrated at 25° C. was placed in the barrel and heat transmitting tube connected to a thermocirculator (25° C.) was wrapped around it. The barrel was connected to a pressure tube connected to a Nitrogen cylinder. Using a series of valves, the system was primed with set-point of 9.5 bar pressure to give 7.5 bar starting pressure. The filtration device was placed over a collection vessel sited on a precision weighing balance that was connected to an Excel computer program. A Bronkhurst pressure controller was used to control and monitor the pressure and when the system valves were opened and timing started, the increasing mass of transfersome filtrate collected on the balance was recorded against the decreasing pressure and increasing time.

The time, pressure, mass data was evaluated in a MathCAD program to determine a P* value. P* is a measure of the activation pressure required for pore penetration and therefore a measure of transfersome membrane stiffness. The average particle size of the transfersomes was measured by photon correlation spectroscopy before and after the CMA filtration.

### Peptide Concentration (CBQCA) Assay

The concentration of the peptide portion of palmitoyl tripeptide-1 with the amino acid sequence glycine-histidine-lysine was measured by derivitisation of the primary amine group of the lysine amino acid with the reagent 3-(4-carboxybenzoyl)quinolone-2-carboxaldehyde (CBQCA) to yield a fluorescent product.

Samples of palmitoyl tripeptide-1 transfersomes and control transfersomes were diluted in a range of 1 in 400 to 1 in 3200 in 0.1 mM sodium borate buffer pH 9.3. Since it was not possible to solubilise palmitoyl tripeptide 1 in aqueous conditions suited to this assay; the determination of concentration of tripeptide 1 in transfersomes was made against a bovine serum albumin (BSA) standard curve. BSA of known concentration was prepared to yield a range of 6.7 μg/ml to 0.33 mg/ml. Derivitisation of the primary amines of the standards and samples was performed in a micro-plate format at room temperature with CBQCA reagent in the presence of potassium cyanide for 1 hour. Measurement was performed by reading with a BMG Fluostar Optima fluorometer with excitation wavelength 485 nm and fluorescence emission wavelength 520 nm.

The fluorescent reading of a blank sample of 0.1 mM sodium borate buffer pH 9.3 was subtracted from all the data. The resulting fluorescent measurement from the BSA standards was analysed with linear regression to produce an equation for the standard curve. The amount of peptide in the palmitoyl tripeptide-1 transfersomes relative to the BSA curve was then determined after subtraction of the fluorescence of the control transfersomes at the equivalent dilution.

### Paper Electrophoresis

The charge characteristics of transfersome preparations were investigated using paper electrophoresis where a paper strip was suspended between two buffer filled reservoirs, the test sample was applied to the strip and an electrical current applied across the strip. Charged particles migrated across the strip, with the direction and distance travelled being determined by the net charge of the particles at the buffer pH.

Volumes (100 μl) of each test sample were applied to the centre of individual 2×20 cm Whatman filter strips (pre-wetted in running buffer; 2.3 mg/ml sodium chloride, 1.5 mg/ml calcium chloride, 1.3 mg/ml glycyl glycine, 25 mg/ml mannitol, 10 mg/ml sucrose and 0.5 mg/ml methionine at pH5.75) and run at 130V for 2 hours. Each strip was stained for PEG (a component of polysorbate 80) with 5% w/v barium chloride and 0.05M iodine and then dried. The extent of travel of the transfersomes away from the centre point for each sample was measured for both the anode and cathode sides of the strip to determine the vesicle net charge.

### Results and Discussion

Results are summarised in Table 5.

TABLE 5
Palmitoyl Peptide Transfersome Analysis

| | Batch | | |
|---|---|---|---|
| | | Palmitoyl | Palmitoyl |
| | Control | Tetrapeptide 7 | Tripeptide 1 |
| Test | Transfersomes | Transfersomes | Transfersomes |
| Photon | | | |
| Correlation | | | |
| Spectrometry: | | | |
| Average Particle | 142.70 | 142.84 | 141.12 |
| Diameter (nm) | | | |
| Polydispersity | 0.071 | 0.053 | 0.062 |
| Index | | | |
| CMA Assay: | | | |
| Filtration % | 14.7 | 14.3 | 14.0 |
| Recovery | | | |
| Deformability | 1.725 | 1.595 | 1.759 |
| P* | | | |
| Average Particle | 74.3 | 72.28 | 74.68 |
| Diameter Post | | | |
| CMA (nm) | | | |
| Polydispersity | 0.11 | 0.12 | 0.099 |
| Index Post CMA | | | |
| Theoretical | 0 mM/ | 0.65 mM/ | 0.65 mM/ |
| Peptide | 0 μg/ml | 296 μg/ml | 221 μg/ml |
| Concentration | | | |
| Peptide | N/A | Non-detectable | Peptide detected |
| Concentration | | due to lack of | (424 μg/ml) |
| (CBQCA | | primary amines | |
| assay) | | in peptide | |
| Paper | Net positive | Net positive | Net positive |
| Electrophoresis | charge | charge | charge |

### Particle Size Measurement

The average particle diameter and polydispersity index were similar for the control transfersomes and for those containing the palmitoyl peptides. This indicated that 10% substitution of polysorbate 80 with a palmitoyl peptide in the transfersomes had not affected the size characteristics.

### Continuous Membrane Adaptability Assay

The deformability P* value was similar for the control transfersomes and for those containing the palmitoyl peptides. The value for the palmitoyl tetrapeptide 7 transfersomes was slightly lower, indicating that 10% substitution of polysorbate 80 with the ester might have had a slight softening effect on the membrane making the vesicles more deformable. However, this was not evidenced in the filtration % recovery which was similar for the palmitoyl peptide transfersomes compared to the control, so the lower P* is possibly not significant.

The average particle diameter post-CMA filtration decreased by almost 50% compared with pre-filtration for both the control transfersomes and for the transfersomes containing the palmitoyl peptide. The polydispersity index was slightly higher, indicating a broader size distribution. These characteristics are as expected for transfersome vesicles.

### Peptide Concentration (CBQCA) Assay

Palmitoyl tetrapeptide 7 transfersomes did not produce a result in the CBQCA assay due to a lack of lysine residues in the sequence to react with the reagent. However, palmitoyl tripeptide 1 was detectable since it contains a lysine. Since it was not possible to solubilise palmitoyl tripeptide 1 in aqueous conditions suited to the assay; the determination of concentration of tripeptide 1 in transfersomes had to be made against a bovine serum albumin (BSA) standard. BSA is a 66 kDa protein with 58 lysine residues; ˜1 per 1138 Da of peptide. The peptide contains 1 lysine in 340 Da. The peptide was detected and an attempt was made to quantify the amount by correcting for the difference in concentration of lysines between BSA and peptide, however the total peptide still appeared to be overestimated; 424 μg/ml compared to theoretical 221 μg/ml.

### Paper Electrophoresis

Control transfersomes and transfersomes containing a palmitoyl peptide all migrated towards the cathode of the electrophoresis apparatus, demonstrating a net positive charge. The presence of palmitoyl tetrapeptide 7 or palmitoyl tripeptide 1 did not therefore alter the charge characteristics of the transfersomes.

### Example Formulation 16 and Manufacture and Testing Thereof

Formulation 16 comprises either phosphatidyl choline (68.700 mg/g) as a lipid, Tween 80 (6.55 mg/g) as a surfactant, naproxen-polysorbate (2.195 mg/g) as an AOI, phosphate (pH 7.7) buffer and ethanol (47.56 mg/g), or phosphatidyl choline (68.700 mg/g) as a lipid, Tween 80 (5.80 mg/g) as a surfactant, diclofenac-polysorbate (2.96 mg/g) as an AOI, phosphate (pH 7.7) buffer and ethanol (47.54 mg/g).

### SUMMARY

Transfersome preparations have successfully been manufactured to contain covalently bonded, non-steroidal anti-inflammatory drugs (NSAIDs); Naproxen and Diclofenac; at 20% polysorbate 80 molar substitution. Test results showed that the size distribution, deformability characteristics and charge of the transfersomes were unaffected by the inclusion of the NSAIDs, that the NSAID esters were accessible on the external surface of the transfersome to a carboxylesterase enzyme and that the NSAID transfersomes had a greater inhibitory effect in a COX-1 enzyme inhibition assay than control transfersomes alone. NSAID transfersomes retained their inhibitory activity after deforming to pass through pores that were smaller than their average size.

### Manufacture

Transfersomes were prepared using soybean phosphatidylcholine (Lipoid SPC S-100) and polysorbate 80, containing either Naproxen-polysorbate 80 ester (Key Organics DK-0035-3) or Diclofenac-polysorbate 80 ester (Key Organics DK-0036-3). A control batch of transfersomes was also made.

### Preparation of NSAID Transfersomes

A 20 g batch of Naproxen-polysorbate transfersomes and a 20 g batch of Diclofenac-polysorbate transfersomes were prepared with soybean phosphatidylcholine: polysorbate 80: NSAID-polysorbate 80 molar ratios of 13.3:0.8:0.2 (accounting for purity of the NSAID-polysorbate 80 esters).

Using gentle heat and stirring, soybean phosphatidylcholine (1.374 g) with EITHER polysorbate 80 (0.131 g) and Naproxen-polysorbate (0.0439 g) OR polysorbate 80 (0.116 g) and Diclofenac-polysorbate (0.0592 g) were dissolved in ethanol to give a total weight of 2.50 g.

Phosphate buffer, pH7.7 (17.50 g) was stirred vigorously at 35° C. while the soybean phosphatidylcholine preparation was added from a syringe fitted with a wide gauge needle. The mixture was stirred for approximately 15 minutes.

The transfersomes were prepared by extrusion through a 0.2 μm filter, followed by a 0.1 μm filter and a further 0.1 μm filter using a Sartorius 47 mm filter system at 35° C. with nitrogen at 4 bar pressure. Each filter had a glass fibre pre-filter on top. Transfersomes were stored in the dark at +5° C.

### Preparation of Control Transfersomes

A 50 g batch of control transfersomes was prepared with a soybean phosphatidylcholine: polysorbate 80 molar ratio of 13.3:1

Using gentle heat and stirring, soybean phosphatidylcholine (3.44 g) and polysorbate 80 (0.425 g) were dissolved in ethanol to give a total weight of 6.26 g.

Phosphate buffer, pH7.7 (43.74 g) was stirred vigorously at 35° C. while the soybean phosphatidylcholine preparation was added from a syringe fitted with a wide gauge needle. The mixture was stirred for approximately 15 minutes.

The control transfersomes were extruded as described for NSAID transfersomes batches. Transfersomes were stored in the dark at +5° C.

### Analytical Methods

### Particle Size Measurement

The average particle size and the particle size distribution for the transfersome preparations were determined by dynamic light scattering using a photon correlation spectrometer.

When coherent light is passed through a suspension of particles, light is scattered in all directions. By measurement and correlation of the scattered light intensity of a particle suspension, it is possible to determine the size and size distribution of the particles in the suspension.

The mean particle size and particle size distribution for each sample were determined using an ALV-5000/E photon correlation spectrometer. Samples were diluted in de-ionised water to give a detectable signal within the range of 50-500 kHz, and then analysed over six measurements, each of 30 seconds duration. The temperature was controlled at 25° C. The data was subjected to a regularised fit cumulative second order analysis to give the mean particle size (reported as r or the mean radius) as well as the particle sizing distribution for the sample (reported as w or width). The mean radius was multiplied by 2 to give the mean diameter (nm).

The polydispersity index (PDI) for each sample was calculated according to the following equation:

$$PDI = {( \frac{w}{r} )}^{2}$$

where: w=width and r=average radius.

### Continuous Membrane Adaptability Assay

The continuous membrane adaptability (CMA) assay used applied pressure to provide activation energy to transfersomes to enable them to deform and pass through a filter pore that is smaller than the average size of the transfersomes.

An Anodisc 13 membrane filter (pore size 20 nm) was mounted on a filtration support in the base of a filtering device and the upper stainless steel barrel was attached. 3 ml transfersome sample pre-equilibrated at 25° C. was placed in the barrel and heat transmitting tube connected to a thermocirculator (25° C.) was wrapped around it. The barrel was connected to a pressure tube connected to a nitrogen cylinder. Using a series of valves, the system was primed with set-point of 9.5 bar pressure to give 7.5bar starting pressure. The filtration device was placed over a collection vessel sited on a precision weighing balance that was connected to an Excel computer program. A Bronkhurst pressure controller was used to control and monitor the pressure and when the system valves were opened and timing started, the increasing mass of transfersome filtrate collected on the balance was recorded against the decreasing pressure and increasing time.

The time, pressure, mass data was evaluated in a MathCAD program to determine a P* value. P* is a measure of the activation pressure required for pore penetration and therefore a measure of transfersome membrane stiffness and deformability. The average particle size of the transfersomes was measured by photon correlation spectroscopy before and after the CMA filtration.

### Carboxylesterase Digest and Rp-HPLC

Release of the tethered non-steroidal anti-inflammatory drugs (NSAIDs); Diclofenac or Naproxen; from the external surface of transfersomes containing polysorbate 80 esters of either of the two compounds was performed by enzymatic digestion of the ester using carboxylesterase 1 isoform B (Sigma E0287). 960 units of enzyme were added per ml of transfersomes, before incubation at +37° C. Samples were taken at 4 hours and the released NSAID extracted by adding 1 volume of acetonitrile/methanol/formic acid (80 v/20 v/0.2 v) followed by sonication for 5 minutes and centrifugation to pellet insoluble components. Supernatant samples were then filtered through a 0.2 μm membrane before diluting 1 in 10 with ultra-high purity water.

Samples were assayed by a reversed phase high pressure liquid chromatography (RP-HPLC) method using a Kinetex C18 5 μm 100 A 4.6×150 mm column and Waters 2695 separation module at +25° C. and a gradient method as per the table below where eluent A was 0.1% trifluoroacetic acid in ultra-high purity water and eluent B was 0.1% trifluoroacetic acid in acetonitrile. Detection for both of the NSAIDs was performed at a wavelength of 254 nm using a Waters 2487 detector.

| Time | Flow | % Eluent | % Eluent |
|---|---|---|---|
| (minutes) | (ml/min) | A | B |
| 0 | 1.2 | 95 | 5 |
| 15 | 1.2 | 5 | 95 |
| 20 | 1.2 | 5 | 95 |
| 21 | 1.2 | 95 | 5 |
| 25 | 1.2 | 95 | 5 |

In addition, a standard curve of each of the NSAIDs in the range of 0.4 to 91 μg/ml was analysed using the same RP-HPLC method. The NSAID peaks were integrated in the resulting chromatograms for the samples and standards. The peak areas of the standards were analysed with linear regression to produce equations for the standard curves. The peak areas for the samples were then used to determine the released NSAID concentration from the equation for the respective standard curve taking into account the dilution from the extraction method. The concentration was compared with the theoretical total NSAID concentration to calculate the % NSAID released from the external surface of the NSAID transfersomes.

### Cyclooxygenase-1 Inhibition Assay

The cyclooxygenase 1 (COX-1) inhibition assay measures the ability of drugs such as NSAIDs to inhibit the activity of the COX-1 enzyme. COX-1 catalyses the conversion of arachidonic acid to prostaglandin H₂. During the reaction the enzyme consumes oxygen. The velocity of oxygen consumption (nmol/ml/min) is a measure of the rate of reaction and is reduced in the presence of inhibitors.

The COX inhibition assay was set up using a Hansatech Oxygraph system that comprised a calibrated Clark oxygen electrode connected to Oxygraph Plus software. A reaction mixture containing 0.1 mM potassium phosphate pH7.2, 2.0 mM phenol, 1 μM hematin was stirred in the reaction chamber at 37° C. until a stable oxygen baseline was attained. 340 units of COX-1 enzyme (Cayman Chemicals CAY60100) was added and allowed to equilibrate for 1 minute before the addition of arachidonic acid substrate. A series of control reactions were performed using arachidonic acid at final concentrations 8, 16, 32 and 6401 For each reaction, the maximum reaction rate was measured on the Oxygraph oxygen curve.

To determine the inhibitory effect of transfersome samples; control, Naproxen or Diclofenac transfersomes were pre-mixed with arachidonic acid for 10 minutes at room temperature prior to the addition of the arachidonic acid mixture to the reaction. The concentrations were chosen so that the final arachidonic acid concentrations in the reaction were 8, 16, 32 and 64 μM.

The arachidonic acid concentration was plotted against the reaction velocity (nmol Oxygen/ml/min) for the control, control transfersomes and NSAID transfersomes reactions and a value was calculated for % inhibition by transfersomes by comparing the reaction velocity at the four substrate concentrations and averaging the decrease in rate. Lineweaver-Burk reciprocal plots (1/arachidonic acid concentration against 1/reaction velocity) were also plotted.

The COX-1 inhibition assay was also performed on samples of transfersomes that had been processed in the continuous membrane adaptability (CMA) assay that used applied pressure to provide activation energy to enable the vesicles to deform and pass through pores that were smaller than their average diameter.

### Paper Electrophoresis

The charge characteristics of transfersome preparations were investigated using paper electrophoresis where a paper strip was suspended between two buffer filled reservoirs, the test sample was applied to the strip and an electrical current applied across the strip. Charged particles migrated across the strip, with the direction and distance travelled being determined by the net charge of the particles at the buffer pH.

Volumes (100 μl) of each test sample were applied to the centre of individual 2×20 cm Whatman filter strips (pre-wetted in running buffer; 2.3 mg/ml sodium chloride, 1.5 mg/ml calcium chloride, 1.3 mg/ml glycyl glycine, 25 mg/ml mannitol, 10 mg/ml sucrose and 0.5 mg/ml methionine at pH5.75) and run at 130V for 2 hours. Each strip was stained for PEG (a component of polysorbate 80) with 5% w/v barium chloride and 0.05M iodine and then dried. The extent of travel of the transfersomes away from the centre point for each sample was measured for both the anode and cathode sides of the strip to determine the vesicle net charge.

### Results and Discussion

Results are summarised in Table 6.

TABLE 6
Control and NSAID Transfersomes Analysis

| | Control | Naproxen | Diclofenac |
|---|---|---|---|
| | Transfersomes | Transfersomes | Transfersomes |
| Photon Correlation | | | |
| Spectrometry: | | | |
| Average Particle | 137.98 | 135.90 | 135.34 |
| Diameter (nm) | | | |
| Polydispersity Index | 0.067 | 0.058 | 0.045 |
| CMA Assay: | | | |
| Filtration % Recovery | 18.0 | 42.3 | 41.8 |
| Deformability P* | 1.632 | 1.321 | 1.339 |
| Average Particle | 72.72 | 77.22 | 73.02 |
| Diameter Post CMA | | | |
| (nm) | | | |
| Polydispersity Index | 0.13 | 0.078 | 0.093 |
| Post CMA | | | |
| Carboxylesterase | | | |
| Digest/HPLC: | | | |
| Theoretical NSAID | 0 mM/ | 1.30 mM/ | 1.30 mM/ |
| Concentration | 0 μg/ml | 299 μg/ml | 385 μg/ml |
| Released External | N/A | 10 μg/ml | 21 μg/ml |
| NSAID Concentration | | (3.3%) | (5.5%) |
| COX-1 Inhibition | | | |
| Assay: | | | |
| Inhibition | 53% | 75% | 62% |
| Inhibition Post-CMA | 33% | 59% | 40% |
| Paper | Net positive | Net positive | Net positive |
| Electrophoresis | charge | charge | charge |

### Particle Size Measurement

The average particle diameter and polydispersity index were similar for the control transfersomes and for those containing an NSAID-polysorbate 80 ester. This indicated that 20% substitution of polysorbate 80 with either Naproxen-polysorbate 80 or Diclofenac-polysorbate in the transfersomes had not affected the size characteristics.

### Continuous Membrane Adaptability Assay

The deformability P* value was slightly lower for the transfersomes containing an NSAID-polysorbate ester than for the control transfersomes, indicating that 20% substitution of polysorbate 80 with either Naproxen-polysorbate 80 or Diclofenac-polysorbate had a slight softening effect on the membrane, making the vesicles more deformable. This was also evidenced in the filtration % recovery which increased for the Naproxen or Diclofenac transfersomes compared to the control.

The average particle diameter post CMA filtration decreased by almost 50% compared with pre-filtration for both the control transfersomes and for the transfersomes containing an NSAID-polysorbate ester. The polydispersity index was slightly higher, indicating a broader size distribution. These characteristics are as expected for transfersome vesicles.

### Carboxylesterase Digest and Rp-HPLC

Incubation of transfersomes containing an NSAID-polysorbate ester with carboxylesterase 1 enzyme resulted in the release of between 3 and 6% of the total NSAID concentration, indicating that a small proportion of the Naproxen or Diclofenac that was tethered to the external surface of the transfersome was accessible to the enzyme.

### Cyclooxygenase-1 Inhibition Assay

Transfersomes containing an NSAID-polysorbate ester inhibited the velocity of reaction of cyclooxygenase 1 (COX-1) enzyme by a greater percentage than control transfersomes. Control transfersomes were expected to inhibit the COX-1 enzyme and tethering known COX-1 inhibitors, Naproxen or Diclofenac, to the external surface of the transfersome has further enhanced that inhibitory effect.

FIGS. 1 and 2 show the arachidonic substrate concentration plotted against the velocity of reaction and the reciprocal (Lineweaver Burk) plots respectively.

Transfersomes that had deformed to pass through a pore that was smaller than their average size in the continuous membrane adaptability (CMA) assay retained the ability to inhibit the COX-1 enzyme.

FIGS. 3 and 4 show the arachidonic substrate concentration plotted against the velocity of reaction and the reciprocal (Lineweaver Burk) plots respectively for the samples post-CMA.

The % inhibition of the COX-1 enzyme was slightly lower post-CMA assay for all 3 transfersome preparations. This was hypothesised to be due to a decrease in the concentration of transfersomes and associated NSAIDs caused by filtration, rather than to a loss in activity of the NSAID. An indication of comparative transfersome concentration was gained from photon correlation spectrometry. The intensity of the frequency signal for the post-CMA samples had decreased in comparison to pre-CMA samples, but was found to be similar for control and NSAID transfersomes, despite the varying filtration recoveries post-CMA.

### Paper Electrophoresis

Control transfersomes and transfersomes containing an NSAID-polysorbate ester all migrated towards the cathode of the electrophoresis apparatus, demonstrating a net positive charge. The presence of Naproxen or Diclofenac did not therefore alter the charge characteristics of the transfersomes.

1-18. (canceled)

19. A vesicle comprising a phospholipid component, a non-ionic surfactant component and a modified component comprising at least one Agent of Interest (AOI), wherein the modified component is a lipid tethered to the AOI or a surfactant tethered to the AOI, or both; and the AOI is tethered such that, when the AOI is on the external surface of the vesicle, a majority of the AOI is external to the vesicular membrane; and the vesicle is deformable to facilitate topical administration of the AOI through the skin of a patient.

20. The vesicle according to claim 19, wherein the modified component is the surfactant tethered to the AOI.

21. The vesicle according to claim 19, wherein the modified component is the lipid tethered to the AOI.

22. The vesicle according to claim 19, wherein the modified component is both the lipid and the surfactant tethered to the AOI.

23. The vesicle according to claim 19, wherein the vesicle comprises a single AOI.

24. The vesicle according to claim 19, wherein the vesicle comprises a plurality of AOIs.

25. The vesicle according to claim 24, wherein the AOIs are homogeneous.

26. The vesicle according to claim 24, wherein the AOIs are heterogeneous.

27. The vesicle according to claim 19, wherein the AOI is selected from the group consisting of an element, an ion, an inorganic salt, a small molecule, an amino acid, a peptide, a protein, a micronutrient, a macromolecule, a macrocyclic molecule and combinations thereof.

28. The vesicle according to claim 19, wherein the AOI is selected from the group consisting of a skin structural protein, a therapeutic protein, a carbohydrate, a chromophore-containing macromolecule, a vitamin, a metal, a metal salt, a non-metallic element, a non-metallic salt, melanin, a melanin analogue, an anti-inflammatory and combinations thereof.

29. The vesicle according to claim 28, wherein the AOI is selected from the group consisting of a vitamin, a metal, a metal salt and combinations thereof.

30. The vesicle according to claim 28, wherein the AOI is a NSAID selected from the group consisting of diclofenac, naproxen and combinations thereof.

31. The vesicle of claim 19, wherein the phospholipid component is phosphatidyl choline, the non-ionic surfactant component is polysorbate 80, and the AOI is selected from the group consisting of ascorbic acid and tripeptide-1.

32. The vesicle of claim 19, wherein the AOI of the modified component is tethered to the lipid of the modified component via at least one lipid glycerol hydroxyl group of the lipid of the modified component by an ester bond.

33. The vesicle of claim 19, wherein the AOI of the modified component is tethered to the lipid of the modified component by replacement of a lipid phosphatidyl moiety of the lipid of the modified component with the AOI such that the lipid of the modified component has two fatty acid chains together with the tethered AOI.

34. The vesicle of claim 19, wherein the AOI is tethered via an ester bond or an amide bond.

35. The vesicle of claim 19, wherein the AOI is tethered via a polymer chain.

36. The vesicle of claim 35, wherein the polymer chain is a polyethylene glycol polymer.

37. A vesicular formulation comprising a plurality of vesicles according to claim 19 and a pharmaceutically acceptable carrier.

38. A method of delivering an AOI through the skin of a patient, the method comprising topically applying to the skin of the patient the vesicular formulation of claim 37 in an amount sufficient to penetrate the skin to deliver the AOI.
